(19) United States
(12) Patent Application Publication
(10) Pub. No.: US 2017/0291875 A1
(43) Pub. Date: Oct. 12, 2017

(54) QUINOLONES AS INHIBITORS OF CLASS IV BROMODOMAIN PROTEINS

(71) Applicant: UCL BUSINESS PLC, London (GB)

(72) Inventors: Paul Vincent Fish, London (GB); Niall Igoe, London (GB); Elliott David Bayle, London (GB)

(21) Appl. No.: 15/508,015

(22) PCT Filed: Aug. 28, 2015
(86) PCT No.: PCT/EP2015/069770

§ 371 Date: Mar. 1, 2017

(30) Foreign Application Priority Data
Sep. 1, 2014 (GB) 1415425.6
Apr. 7, 2015 (GB) 1505911.6

(51) Int. Cl. C07D 215/36; A61K 31/4704; G01N 33/574; C07D 215/38

(52) U.S. Cl. CPC C07D 215/36; C07D 215/38; A61K 31/4704; G01N 33/574; C12Q 2600/158; G01N 33/48

(57) ABSTRACT

The present invention provides compounds of formula (I) as described herein and pharmaceutically acceptable salts, hydrates and solvates thereof for use in medicine, for example in the treatment of acute myeloid leukaemia:

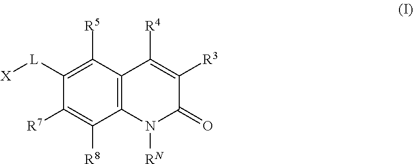

### RELATED APPLICATIONS

This application is related to United Kingdom patent application number 1415425.6 filed 1 Sep. 2014 and United Kingdom patent application number 1505911.6 filed 7 Apr. 2015, the contents of each of which are incorporated herein by reference in their entirety.

### TECHNICAL FIELD

The present invention pertains generally to the field of therapeutic compounds, and more specifically to certain substituted quinolone compounds.

The present invention also pertains to pharmaceutical compositions comprising such compounds, to the use of such compounds and compositions, in vitro or in vivo, to kill cells and/or inhibit cell proliferation, to the use of such compounds and compositions to treat proliferative disorders such as cancer, and to methods for their preparation.

### BACKGROUND

The human BRPF (bromodomain and PHD finger containing) family of histone acyl-lysine reader proteins (BRPF-1, -2 and -3) are important regulators of epigenetic signaling. These proteins recognize specific acyl lysine residues on histones, leading to changes in chromatin structure, multi-protein complex formation and transcriptional regulation.

In particular, BRPF1 is a unique epigenetic regulator containing multiple structural domains for recognizing different chromatin modifications and possesses sequence motifs for forming multiple complexes with three different histone acyltransferases: MOZ, MORF and HBO1, also known as lysine acetyltransferase 6A (KAT6A), KAT6B and KAT7, respectively. Within these complexes, BRPF1 serves as a scaffold for bridging subunit interaction, stimulating acetyltransferase activity, governing substrate specificity and stimulating gene expression.¹

For example some BRPF complexes have been found to upregulate HOX (homeobox) genes mediated by histone deacetylation.²

There is an emerging understanding of the potential role of bromodomain proteins in acute myeloid leukemia (AML).^{2,3} Without wishing to be bound by theory, it is though that the activation of the BRPF1/HOX pathway through MOZ histone acyl transfer is critical for MOZ-TIF2 to induce AML. Acute myeloid leukemia (AML) is a life-threatening stem cell neoplasm that affects myeloid cells. It is a complex disease shown to be highly heterogeneous at both genetic and biological levels (>100 mutations)

The role of BRPF1 and other bromodomain proteins in other cancers and non-cancer indications is also being explored. The role of HOX gene expression/loss, and of BRPF complexes with lysine acyl transferases MOZ, MORF and HBO1, are of interest in the context of a number of disease indications.

For example, altered HOX gene expression may contribute to the development of pulmonary diseases, such as primary pulmonary hypertension (PPH) and emphysema.⁴ Other studies have suggested that HOX genes are involved in tumorigenesis, particularly in the lung.^{5,6}

The MOZ and MORF genes are mutated in cancers such as leukemia, as well as in multiple developmental disorders characterized by intellectual disability and/or associated with psychiatric illnesses such as schizophrenia (e.g. DiGeorge syndrome, Noonan syndrome-like disorder, Ohdo syndrome, genitopatellar syndrome, blepharophimosis-ptosis-epicanthus inversus syndrome).^{7,8} A role for BRFP1 in neurological development has been proposed,^{8,9} as well as a crucial role in embryo development and cell cycle control.⁷

In the context of the above-noted research, various potential medical uses of selective BRPF inhibitors are supported by the role of modulation of HOX gene expression/loss and through the role of BRPF complexes with lysine acyl transferases MOZ, MORF and HBO1.

WO2013/027168 (Pfizer, Inc) discloses certain heterocyclic compounds as inhibitors of the BET family of bromodomain inhibitors, specifically of bromodomain-containing protein 4 (BRD4). The BET family of bromodomain proteins is distinct from the class IV bromodomains discussed above.

### General Notes

A number of patents and publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

This disclosure includes information that may be useful in understanding the present invention. It is not an admission that any of the information provided herein is prior art or relevant to the presently claimed invention, or that any publication specifically or implicitly referenced is prior art.

### SUMMARY OF THE INVENTION

In light of the above discussion, it can be seen that the development of novel compounds and compositions which selectively inhibit class IV bromodomain proteins would be a contribution to the art.

The present inventors have developed a novel class of substituted quinolone compounds with potent and selective activity against BRPF1 and other class IV bromodomain proteins.

Accordingly, one aspect of the present invention pertains to certain such quinolone compounds, as further described herein.

Another aspect of the invention pertains to compositions (e.g., a pharmaceutical compositions) comprising a compound of the invention as described herein and a pharmaceutically acceptable carrier or diluent.

Another aspect of the invention pertains to methods of preparing a composition (e.g., a pharmaceutical composition) comprising the step of admixing a compound of the invention as described herein and a pharmaceutically acceptable carrier or diluent.

Another aspect of the present invention pertains to methods of treatment comprising administering to a subject in need of treatment a therapeutically-effective amount of a compound of the invention as described herein, preferably in the form of a pharmaceutical composition.

Another aspect of the present invention pertains to a compound of the invention as described herein for use in a method of treatment of the human or animal body by therapy.

Another aspect of the present invention pertains to use of a compound of the invention as described herein, in the manufacture of a medicament for use in treatment.

In some embodiments, the treatment is treatment of a proliferative disorder.

In some embodiments, the treatment is treatment of cancer, in particular a cancer characterised by activation of the BRPF1/HOX pathway.

In some embodiments, the treatment is treatment of acute myeloid leukemia (AML).

Another aspect of the present invention pertains to a kit comprising (a) a compound of the invention as described herein, preferably provided as a pharmaceutical composition and in a suitable container and/or with suitable packaging; and (b) instructions for use, for example, written instructions on how to administer the compound.

Another aspect of the present invention pertains to certain methods of synthesis, as described herein.

Another aspect of the present invention pertains to a compound (e.g., a compound of the invention) obtainable by a method of synthesis as described herein, or a method comprising a method of synthesis as described herein.

Another aspect of the present invention pertains to a compound (e.g., a compound of the invention) obtained by a method of synthesis as described herein, or by a method comprising a method of synthesis as described herein.

As will be appreciated by one of skill in the art, features and preferred embodiments of one aspect of the invention will also pertain to other aspect of the invention.

### DETAILED DESCRIPTION OF THE INVENTION

### Compounds

One aspect of the present invention pertains to compounds as described in more detail in the numbered paragraphs below and to salts, hydrates, and solvates thereof (e.g., pharmaceutically acceptable salts, hydrates, and solvates thereof).

A compound of general formula I:

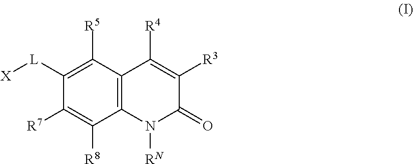

wherein:
R³ is selected from —R^{3A} and —OR^{3B} wherein R^{3A} and R^{3B} are each independently selected from hydrogen, C₁₋₄alkyl and C₁₋₄haloalkyl;
R⁴ is selected from —R^{4A} and —OR^{4B} wherein R^{4A} and R^{4B} are each independently selected from hydrogen, C₁₋₄alkyl and C₁₋₄haloalkyl;
R⁵ is selected from —R^{5A} and —OR^{5B} wherein R^{5A} is independently selected from hydrogen, halo, C₁₋₄alkyl, C₂₋₄alkenyl, C₂₋₄alkynyl, C₃₋₆ cycloalkyl and C₁₋₄haloalkyl, and wherein R^{5B} is independently selected from hydrogen, C₁₋₄alkyl, C₃₋₆ cycloalkyl and C₁₋₄haloalkyl;
R⁷ is selected from —R^{7A} and —OR^{7B} wherein R^{7A} and R^{7B} are each independently selected from hydrogen, C₁₋₄alkyl, C₃₋₆cycloalkyl, and C₁₋₄haloalkyl;

R⁸ is selected from —R^{8A} and —OR^{8B} wherein R^{8A} is independently selected from hydrogen, halo, C₁₋₄alkyl, and C₁₋₄haloalkyl, and wherein R^{8B} is independently selected from hydrogen, C₁₋₄alkyl and C₁₋₄haloalkyl;
R^{N} is selected from C₁₋₄alkyl, C₁₋₄haloalkyl, R^{Z}, and —Z^{N}—R^{Z} wherein Z^{N} is C₁₋₄alkylene and each R^{Z} is independently C₃₋₆cycloalkyl;
L is a sulfonamide linker;
X is selected from aryl, C₁₋₆alkyl, and C₃₋₆cycloalkyl, and is optionally substituted.
   Groups R³ to R⁷

### R³

R³ is selected from —R^{3A} and —OR^{3B} wherein R^{3A} and R^{3B} are each independently selected from hydrogen, C₁₋₄alkyl and C₁₋₄haloalkyl.

A compound according to paragraph [0001] wherein R³ is —R^{3A}.

A compound according to paragraph [0002] wherein R^{3A} is independently hydrogen.

A compound according to paragraph [0002] wherein R^{3A} is independently C₁₋₄alkyl.

A compound according to paragraph [0004] wherein R^{3A} is -Me, -Et, -nPr, -iPr, -nBu, -iBu, or -tBu.

A compound according to paragraph [0004] wherein R^{3A} is -Me or -Et.

A compound according to paragraph [0004] wherein R^{3A} is -Me.

A compound according to paragraph [0002] wherein R^{3A} is independently C₁₋₄haloalkyl.

As used herein, the term ‘C₁₋₄haloalkyl’ refers to a C₁₋₄alkyl group which is substituted with one or more halo (i.e., —F, —Cl, —Br, —I) substituents; corresponding terms such as ‘C₁₋₄fluoroalkyl’ shall be interpreted accordingly.

A compound according to paragraph [0008] wherein R^{3A} is C₁₋₄fluoroalkyl.

A compound according to paragraph [0009] wherein R^{3A} is selected from:
—CF₃, —CHF₂, —CH₂F
—CH₂CF₃, —CH₂CH₂F, —CH₂CHF₂
—C(H)FCF₃, —C(H)FCH₂F, —C(H)FCHF₂
—CF₂CF₃, —CF₂CHF₂, —CF₂CH₂F.

A compound according to paragraph [0009] wherein R^{3A} is selected from:
—CF₃, —CHF₂, —CH₂F.

A compound according to paragraph [0009] wherein R^{3A} is —CF₃.

A compound according to paragraph [0001] wherein R³ is —OR^{3B}.

A compound according to paragraph [0013] wherein R^{3B} is independently hydrogen.

A compound according to paragraph [0013] wherein R^{3B} is independently C₁₋₄alkyl.

A compound according to paragraph [0015] wherein R^{3B} is -Me, -Et, -nPr, -iPr, -nBu, -iBu, or -tBu.

A compound according to paragraph [0015] wherein R^{3B} is -Me or -Et.

A compound according to paragraph [0015] wherein R^{3B} is -Me.

A compound according to paragraph [0013] wherein R^{3B} is independently C₁₋₄ haloalkyl.

A compound according to paragraph [0019] wherein R^{3B} is C₁₋₄fluoroalkyl.

A compound according to paragraph [0020] wherein R^{3B} is selected from:
—CF₃, —CHF₂, —CH₂F
—CH₂CF₃, —CH₂CH₂F, —CH₂CHF₂
—C(H)FCF₃, —C(H)FCH₂F, —C(H)FCHF₂
—CF₂CF₃, —CF₂CHF₂, —CF₂CH₂F.

A compound according to paragraph [0020] wherein R^{3B} is selected from:
—CF₃, —CHF₂, —CH₂F.

A compound according to paragraph [0020] wherein R^{3B} is —CF₃.

### R⁴

R⁴ is selected from —R^{4A} and —OR^{4B} wherein R^{4A} and R^{4B} are each independently selected from hydrogen, C₁₋₄alkyl and C₁₋₄haloalkyl.

A compound according to any one of paragraphs [0001] to [0023] wherein R⁴ is —R^{4A}.

A compound according to paragraph [0024] wherein R^{4A} is independently hydrogen.

A compound according to paragraph [0024] wherein R^{4A} is independently C₁₋₄alkyl.

A compound according to paragraph [0026] wherein R^{4A} is -Me, -Et, -nPr, -iPr, -nBu, -iBu, or -tBu.

A compound according to paragraph [0026] wherein R^{4A} is -Me or -Et.

A compound according to paragraph [0026] wherein R^{4A} is -Me.

A compound according to paragraph [0024] wherein R^{4A} is independently C₁₋₄ haloalkyl.

A compound according to paragraph [0030] wherein R^{4A} is C₁₋₄fluoroalkyl.

A compound according to paragraph [0031] wherein R^{4A} is selected from:
—CF₃, —CHF₂, —CH₂F
—CH₂CF₃, —CH₂CH₂F, —CH₂CHF₂
—C(H)FCF₃, —C(H)FCH₂F, —C(H)FCHF₂
—CF₂CF₃, —CF₂CHF₂, —CF₂CH₂F.

A compound according to paragraph [0031] wherein R^{4A} is selected from:
—CF₃, —CHF₂, —CH₂F.

A compound according to paragraph [0031] wherein R^{4A} is —CF₃.

A compound according to any one of paragraphs [0001] to [0023] wherein R⁴ is —OR^{4B}.

A compound according to paragraph [0035] wherein R^{4B} is independently hydrogen.

A compound according to paragraph [0035] wherein R^{4B} is independently C₁₋₄alkyl.

A compound according to paragraph [0037] wherein R^{4B} is -Me, -Et, -nPr, -iPr, -nBu, -iBu, or -tBu.

A compound according to paragraph [0037] wherein R^{4B} is -Me or -Et.

A compound according to paragraph [0037] wherein R^{4B} is -Me.

A compound according to paragraph [0035] wherein R^{4B} is independently C₁₋₄ haloalkyl.

A compound according to paragraph [0041] wherein R^{4B} is C₁₋₄fluoroalkyl.

A compound according to paragraph [0042] wherein R^{4B} is selected from:
—CF₃, —CHF₂, —CH₂F
—CH₂CF₃, —CH₂CH₂F, —CH₂CHF₂
—C(H)FCF₃, —C(H)FCH₂F, —C(H)FCHF₂
—CF₂CF₃, —CF₂CHF₂, —CF₂CH₂F.

A compound according to paragraph [0042] wherein R^{4B} is selected from:
—CF₃, —CHF₂, —CH₂F.

A compound according to paragraph [0042] wherein R^{4B} is —CF₃.

### R⁵

R⁵ is selected from —R^{5A} and —OR^{5B} wherein R^{5A} is independently selected from hydrogen, halo, C₁₋₄alkyl, C₂₋₄alkenyl, C₂₋₄alkynyl, C₃₋₆ cycloalkyl and C₁₋₄haloalkyl, and wherein R^{5B} is independently selected from hydrogen, C₁₋₄alkyl, C₃₋₆ cycloalkyl and C₁₋₄haloalkyl.

A compound according to any one of paragraphs [0001] to [0045] wherein R⁵ is —R^{5A}.

A compound according to paragraph [0046] wherein R^{5A} is independently hydrogen.

A compound according to paragraph [0046] wherein R^{5A} is independently halo.

A compound according to paragraph [0048] wherein R^{5A} is —F, —Cl, —Br or —I.

A compound according to paragraph [0048] wherein R^{5A} is selected from —F and —Cl.

A compound according to paragraph [0048] wherein R^{5A} is —F.

A compound according to paragraph [0046] wherein R^{5A} is independently C₁₋₄alkyl.

A compound according to paragraph [0052] wherein R^{5A} is -Me, -Et, -nPr, -iPr, -nBu, -iBu, or -tBu.

A compound according to paragraph [0052] wherein R^{5A} is -Me or -Et.

A compound according to paragraph [0052] wherein R^{5A} is -Me.

A compound according to paragraph [0046] wherein R^{5A} is independently C₂₋₄ alkenyl.

A compound according to paragraph [0052] wherein R^{5A} is selected from:
—CH═CH₂,
—CH═CH—CH₃, —CH₂—CH═CH₂,
—CH═CH—CH₂CH₃, —CH₂—CH═CHCH₃, —CH₂—CH₂CH═CH₂,
—CH═C(CH₃)₂, and —C(CH₃)═C(H)CH₃

A compound according to paragraph [0048] wherein R^{5A} is selected from:
—CH═CH₂ and
—CH₂—CH═CH₂.

A compound according to paragraph [0046] wherein R^{5A} is independently C₂₋₄ alkynyl.

A compound according to paragraph [0054] wherein R^{5A} is selected from:
—C≡CH,
—C≡CCH₃, —CH₂C≡CH,
—C≡CCH₂CH₃, —CH₂C≡CCH₃, —CH₂CH₂C≡CH.

A compound according to paragraph [0054] wherein R^{5A} is —C≡CH.

A compound according to paragraph [0046] wherein R^{5A} is independently C₃₋₆cycloalkyl.

A compound according to paragraph [0057] wherein R^{5A} is cyclopropyl, cyclobutyl, cyclopentyl, or cyclohexyl.

A compound according to paragraph [0057] wherein R^{5A} is cyclopropyl:

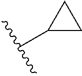 [SMILES: *C1CC1]

A compound according to paragraph [0046] wherein R^{5A} is independently C₁₋₄haloalkyl.

A compound according to paragraph [0060] wherein R^{5A} is C₁₋₄fluoroalkyl.

A compound according to paragraph [0061] wherein R^{5A} is selected from:
—CF₃, —CHF₂, —CH₂F
—CH₂CF₃, —CH₂CH₂F, —CH₂CHF₂
—C(H)FCF₃, —C(H)FCH₂F, —C(H)FCHF₂
—CF₂CF₃, —CF₂CHF₂, —CF₂CH₂F.

A compound according to paragraph [0061] wherein R^{5A} is selected from:
—CF₃, —CHF₂, —CH₂F.

A compound according to paragraph [0061] wherein R^{5A} is —CF₃.

A compound according to any one of paragraphs [0001] to [0045] wherein R⁵ is —OR^{5B}.

A compound according to paragraph [0065] wherein R^{5B} is independently hydrogen.

A compound according to paragraph [0065] wherein R^{5B} is independently C₁₋₄alkyl.

A compound according to paragraph [0067] wherein R^{5B} is -Me, -Et, -nPr, -iPr, -nBu, -iBu, or -tBu.

A compound according to paragraph [0067] wherein R^{5B} is -Me or -Et.

A compound according to paragraph [0067] wherein R^{5B} is -Me.

A compound according to paragraph [0065] wherein R^{5B} is independently C₃₋₆cycloalkyl.

A compound according to paragraph [0071] wherein R^{5B} is cyclopropyl, cyclobutyl, cyclopentyl, or cyclohexyl.

A compound according to paragraph [0071] wherein R^{5B} is cyclopropyl:

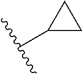 [SMILES: *C1CC1]

A compound according to paragraph [0065] wherein R^{5B} is independently C₁₋₄haloalkyl.

A compound according to paragraph [0074] wherein R^{5B} is C₁₋₄fluoroalkyl.

A compound according to paragraph [0075] wherein R^{5B} is selected from:
—CF₃, —CHF₂, —CH₂F
—CH₂CF₃, —CH₂CH₂F, —CH₂CHF₂
—C(H)FCF₃, —C(H)FCH₂F, —C(H)FCHF₂
—CF₂CF₃, —CF₂CHF₂, —CF₂CH₂F.

A compound according to paragraph [0075] wherein R^{5B} is selected from:
—CF₃, —CHF₂, —CH₂F.

A compound according to paragraph [0075] wherein R^{5B} is —CF₃.

### R⁷

R⁷ is selected from —R^{7A} and —OR^{7B} wherein R^{7A} is independently selected from hydrogen, C₁₋₄alkyl and C₁₋₄haloalkyl and R^{7B} is independently selected from hydrogen, C₁₋₄alkyl, C₃₋₆ cycloalkyl and C₁₋₄haloalkyl.

A compound according to any one of paragraphs [0001] to [0078] wherein R⁷ is —R^{7A}.

A compound according to paragraph [0079] wherein R^{7A} is independently hydrogen.

A compound according to paragraph [0079] wherein R^{7A} is independently C₁₋₄alkyl.

A compound according to paragraph [0081] wherein R^{7A} is independently -Me, -Et, -nPr, -iPr, -nBu, -iBu, or -tBu.

A compound according to paragraph [0081] wherein R^{7A} is -Me or -Et.

A compound according to paragraph [0081] wherein R^{7A} is -Me.

A compound according to paragraph [0079] wherein R^{7A} is independently C₁₋₄haloalkyl.

A compound according to paragraph [0085] wherein R^{7A} is C₁₋₄fluoroalkyl.

A compound according to paragraph [0086] wherein R^{7A} is selected from:
—CF₃, —CHF₂, —CH₂F
—CH₂CF₃, —CH₂CH₂F, —CH₂CHF₂
—C(H)FCF₃, —C(H)FCH₂F, —C(H)FCHF₂
—CF₂CF₃, —CF₂CHF₂, —CF₂CH₂F.

A compound according to paragraph [0086] wherein R^{7A} is selected from:
—CF₃, —CHF₂, —CH₂F.

A compound according to paragraph [0086] wherein R^{7A} is —CF₃.

A compound according to any one of paragraphs [0001] to [0078] wherein R⁷ is —OR^{7B}

A compound according to paragraph [0090] wherein R^{7B} is independently hydrogen.

A compound according to paragraph [0090] wherein R^{7B} is independently C₁₋₄alkyl.

A compound according to paragraph [0092] wherein R^{7B} is independently -Me, -Et, -nPr, -iPr, -nBu, -iBu, or -tBu.

A compound according to paragraph [0092] wherein R^{7B} is -Me or -Et.

A compound according to paragraph [0092] wherein R^{7B} is -Me.

A compound according to paragraph [0090] wherein R^{7B} is independently C₃₋₆cycloalkyl.

A compound according to paragraph [0096] wherein R^{7B} is cyclopropyl, cyclobutyl, cyclopentyl, or cyclohexyl.

A compound according to paragraph [0096] wherein R^{7B} is cyclopropyl:

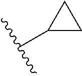 [SMILES: *C1CC1]

A compound according to paragraph [0090] wherein R^{7B} is independently C₁₋₄haloalkyl.

A compound according to paragraph [0099] wherein R^{7B} is C₁₋₄fluoroalkyl.

A compound according to paragraph [0100] wherein R^{7B} is selected from:
—CF₃, —CHF₂, —CH₂F
—CH₂CF₃, —CH₂CH₂F, —CH₂CHF₂
—C(H)FCF₃, —C(H)FCH₂F, —C(H)FCHF₂
—CF₂CF₃, —CF₂CHF₂, —CF₂CH₂F.

A compound according to paragraph [0100] wherein R^{7B} is selected from:
—CF₃, —CHF₂, —CH₂F.

A compound according to paragraph [0100] wherein R^{7B} is —CF₃.

### R⁸

R⁸ is selected from —R^{8A} and —OR^{8B} wherein R^{8A} is independently selected from hydrogen, halo, C₁₋₄alkyl, and C₁₋₄haloalkyl, and wherein R^{8B} is independently selected from hydrogen, C₁₋₄alkyl and C₁₋₄haloalkyl.

A compound according to any one of paragraphs [0001] to [0103] wherein R⁸ is —R^{8A}.

A compound according to paragraph [0104] wherein R^{8A} is independently hydrogen.

A compound according to paragraph [0104] wherein R^{8A} is independently halo.

A compound according to paragraph [0106] wherein R^{8A} is —F, —Cl, —Br or —I.

A compound according to paragraph [0106] wherein R^{8A} is selected from —F and —Cl.

A compound according to paragraph [0106] wherein R^{8A} is —F.

A compound according to paragraph [0104] wherein R^{8A} is independently C₁₋₄alkyl.

A compound according to paragraph [0110] wherein R^{8A} is independently -Me, -Et, -nPr, -iPr, -nBu, -iBu, or -tBu.

A compound according to paragraph [0110] wherein R^{8A} is -Me or -Et.

A compound according to paragraph [0110] wherein R^{8A} is -Me.

A compound according to paragraph [0104] wherein R^{8A} is independently C₁₋₄haloalkyl.

A compound according to paragraph [0114] wherein R^{8A} is C₁₋₄fluoroalkyl.

A compound according to paragraph [0115] wherein R^{8A} is selected from:
—CF₃, —CHF₂, —CH₂F
—CH₂CF₃, —CH₂CH₂F, —CH₂CHF₂
—C(H)FCF₃, —C(H)FCH₂F, —C(H)FCHF₂
—CF₂CF₃, —CF₂CHF₂, —CF₂CH₂F.

A compound according to paragraph [0115] wherein R^{8A} is selected from:
—CF₃, —CHF₂, —CH₂F.

A compound according to paragraph [0115] wherein R^{8A} is —CF₃.

A compound according to any one of paragraphs [0001] to [0103] wherein R⁸ is —OR⁸B

A compound according to paragraph [0119] wherein R^{8B} is independently hydrogen.

A compound according to paragraph [0119] wherein R^{8B} is independently C₁₋₄alkyl.

A compound according to paragraph [0121] wherein R^{8B} is independently -Me, -Et, -nPr, -iPr, -nBu, -iBu, or -tBu.

A compound according to paragraph [0121] wherein R^{8B} is -Me or -Et.

A compound according to paragraph [0121] wherein R^{8B} is -Me.

A compound according to paragraph [0119] wherein R^{8B} is independently C₃₋₆ cycloalkyl.

A compound according to paragraph [0125] wherein R^{8B} is cyclopropyl, cyclobutyl, cyclopentyl, or cyclohexyl.

A compound according to paragraph [0125] wherein R^{8B} is cyclopropyl:

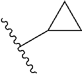 [SMILES: *C1CC1]

A compound according to paragraph [0119] wherein R^{8B} is independently C₁₋₄haloalkyl.

A compound according to paragraph [0128] wherein R^{8B} is C₁₋₄fluoroalkyl.

A compound according to paragraph [0129] wherein R^{8B} is selected from:
—CF₃, —CHF₂, —CH₂F
—CH₂CF₃, —CH₂CH₂F, —CH₂CHF₂
—C(H)FCF₃, —C(H)FCH₂F, —C(H)FCHF₂
—CF₂CF₃, —CF₂CHF₂, —CF₂CH₂F.

A compound according to paragraph [0129] wherein R^{8B} is selected from:
—CF₃, —CHF₂, —CH₂F.

A compound according to paragraph [0129] wherein R^{8B} is —CF₃.

### Group R^{N}

R^{N} is selected from C₁₋₄alkyl, C₁₋₄haloalkyl, R^{Z}, and —Z^{N}—R^{Z} wherein Z^{N} is C₁₋₄alkylene and each R^{Z} is independently C₃₋₆cycloalkyl.

A compound according to any one of paragraphs [0001] to [0132] wherein R^{N} is independently C₁₋₄alkyl.

A compound according to paragraph [0133] wherein R^{N} is selected from -Me, -Et, -nPr, -iPr, -nBu, -iBu, and -tBu.

A compound according to paragraph [0133] wherein R^{N} is -Me or -Et.

A compound according to paragraph [0133] wherein R^{N} is -Me.

A compound according to paragraph [0133] wherein R^{N} is -Et.

A compound according to any one of paragraphs [0001] to [0132] wherein R^{N} is independently C₁₋₄haloalkyl.

A compound according to paragraph [0138] wherein R^{N} is C₁₋₄fluoroalkyl.

A compound according to paragraph [0139] wherein R^{N} is selected from:
—CF₃, —CHF₂, —CH₂F
—CH₂CF₃, —CH₂CH₂F, —CH₂CHF₂
—C(H)FCF₃, —C(H)FCH₂F, —C(H)FCHF₂
—CF₂CF₃, —CF₂CHF₂, —CF₂CH₂F.

A compound according to paragraph [0139] wherein R^{N} is selected from:
—CF₃, —CHF₂, —CH₂F.

A compound according to paragraph [0139] wherein R^{N} is —CF₃.

A compound according to any one of paragraphs [0001] to [0132] wherein R^{N} is R^{Z} or —Z^{N}—R^{Z}, wherein Z^{N} is C₁₋₄alkylene and R^{Z} is C₃₋₆cycloalkyl.

A compound according to paragraph [0142] wherein R^{N} is R^{Z}.

A compound according to paragraph [0142] wherein R^{N} is —Z^{N}—R^{Z}.

A compound according to paragraph [0143] or paragraph [0144] wherein R^{Z} is C₃₋₅ cycloalkyl.

A compound according to paragraph [0145] wherein R^{Z} is cyclopropyl, cyclobutyl, or cyclopentyl.

A compound according to paragraph [0145] wherein R^{N} is cyclopropyl:

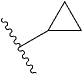 [SMILES: *C1CC1]

A compound according to paragraph [0144] wherein Z^{N} is selected from:
—CH₂—,
—CH₂CH₂—, —CH(CH₃)—,
—CH₂CH₂CH₂—, —CH(CH₃)CH₂—, —CH₂CH(CH₃)—, —CH(CH₂CH₃)—,
—CH₂CH₂CH₂CH₂—, —CH(CH₃)CH₂CH₂—, —CH₂CH(CH₃)CH₂—, —CH₂CH₂CH(CH₃)—,
—CH(CH₂CH₃)CH₂—, —CH₂CH(CH₂CH₃)—, —CH(CH₂CH₂CH₃)—.

A compound according to paragraph [0144] wherein Z^{N} is selected from:
—CH₂—, —CH₂CH₂—, —CH₂CH₂CH₂—, —CH₂CH₂CH₂CH₂—.

### Linker L

L is a sulfonamide linker.

A compound according to any one of paragraphs [0001] to [0149] wherein L is a sulfonamide linker selected from:

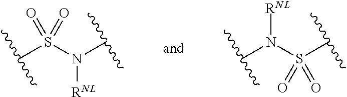

wherein R^{NL} is selected from hydrogen and C₁₋₄alkyl.

A compound according to paragraph [0150] wherein L is:

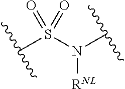

i.e. a compound of formula (IIa):

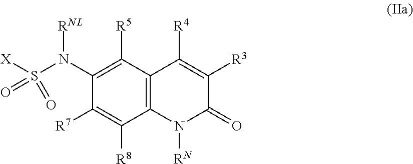

A compound according to paragraph [0150] wherein L is:

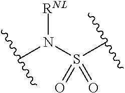

i.e. a compound of formula (IIb):

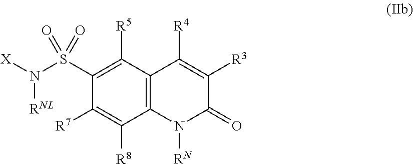

### R^{NL}

A compound according to paragraph [0151] or [0152] wherein R^{NL} is hydrogen.

A compound according to paragraph [0151] or [0152] wherein R^{NL} is C₁₋₄alkyl.

A compound according to paragraph [0154] wherein R^{NL} is independently -Me, -Et, -nPr, -iPr, -nBu, -iBu, or -tBu.

A compound according to paragraph [0154] wherein R^{NL} is -Me or -Et.

A compound according to paragraph [0154] wherein R^{NL} is -Me.

### Group X

X is selected from aryl, C₁₋₆alkyl and C₃₋₆ cycloalkyl, and is optionally substituted.

A compound according to any one of paragraphs [0001] to [0157] wherein X is unsubstituted.

A compound according to any one of paragraphs [0001] to [0157] wherein X is optionally substituted with one or more substituents R^{X} wherein each R^{X} is independently selected from halo, C₁₋₄alkyl, C₁₋₄haloalkyl, —OR^{XO}, —C(═O)OR^{XO}, —N(R^{XN})₂, —C(═O)N(R^{XN})₂, —N(R^{XN})C(═O)R^{XN}, —SR^{XS}, —S(═O)R^{XS}, —S(═O)₂R^{XS}, —SO₂OR^{XO}, —SO₂N(R^{XN})₂, —CN, —NO₂, and aryl; wherein said aryl is optionally substituted with one or more substituents R^{XX}, wherein R^{XX} is selected from halo, C₁₋₄alkyl, C₁₋₄haloalkyl, aryl, —OR^{XO}, —C(═O)OR^{XO}, —N(R^{XN})₂, —C(═O)N(R^{XN})₂, —N(R^{XN})C(═O)R^{XN}, —SR^{XS}, —S(═O)R^{XS}, —S(═O)₂R^{XS}, —SO₂OR^{XO}, —SO₂N(R^{XN})₂, —CN, and —NO₂; and wherein each R^{XO}, R^{XN} and R^{XS} is independently selected from hydrogen, C₁₋₄alkyl and C₁₋₄haloalkyl.

A compound according to paragraph [0158] or [0159] wherein X is aryl.

A compound according to paragraph [0160] wherein X is selected from C₆₋₂₀carboaryl and C₆₋₁₂ heteroaryl.

A compound according to paragraph [0161] wherein X is selected from phenyl, naphthyl, furanyl, thienyl, pyrrolyl, imidazolyl, pyrazolyl, triazolyl, tetrazolyl, oxazolyl, isoxazolyl, thiazolyl, isothiazolyl, pyridyl, pyrimidinyl, pyridazinyl, indolyl, isoindolyl, benzofuranyl, isobenzofuranyl, benzothienyl, isobenzothienyl, indazolyl, benzimidazolyl, benzothiazolyl, benzoisothiazolyl, benzoxazolyl, benzoisoxazolyl, quinolinyl, isoquinolinyl, cinnolinyl, or quinazolinyl, and is optionally substituted.

A compound according to paragraph [0161] wherein X is phenyl.

A compound according to paragraph [0161] wherein X is C₅₋₆ heteroaryl.

A compound according to paragraph [0164] wherein X is selected from furanyl, thienyl, pyrrolyl, imidazolyl, pyrazolyl, triazolyl, tetrazolyl, oxazolyl, isoxazolyl, thiazolyl, isothiazolyl, pyridyl, pyrimidinyl, or pyridazinyl.

A compound according to paragraph [0164] wherein X is selected from thienyl and pyridyl.

A compound according to paragraph [0158] or [0159] wherein X is C₁₋₆alkyl.

A compound according to paragraph [0167] wherein X is C₁₋₄alkyl.

A compound according to paragraph [0168] wherein X is selected from -Me, -Et, -nPr, -iPr, -nBu, -iBu, -tBu.

A compound according to paragraph [0168] wherein X is selected from -Me, -Et, and -iPr.

A compound according to paragraph [0158] or [0159] wherein X is independently C₃₋₆ cycloalkyl.

A compound according to paragraph [0171] wherein X is cyclopropyl, cyclobutyl, cyclopentyl, or cyclohexyl.

A compound according to paragraph [0171] wherein X is cyclopropyl or cyclohexyl.

A compound according to paragraph [0173] wherein X is cyclopropyl:

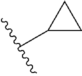 [SMILES: *C1CC1]

A compound according to paragraph [0173] wherein X is cyclohexyl:

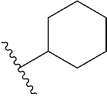 [SMILES: *C1CCCCC1]

### R^{X}

X is optionally substituted, for example with one or more substituents R^{X}.

A compound according to any one of paragraphs [0111] to [0175] wherein X is substituted with at least one substituent R^{X} wherein R^{X} is independently selected from halo, C₁₋₄alkyl, C₁₋₄haloalkyl, —OR^{XO}, —C(═O)OR^{XO}, —N(R^{XN})₂, —C(═O)N(R^{XN})₂, —SR^{XS}, —S(═O)R^{XS}, —S(═O)₂R^{XS}, —SO₂OR^{XO}, —SO₂N(R^{XN})₂, —CN, —NO₂ and aryl; wherein said aryl is optionally substituted with one or more substituents R^{XX}, wherein R^{XX} is selected from halo, C₁₋₄alkyl, C₁₋₄haloalkyl, aryl, —OR^{XO}, —C(═O)OR^{XO}, —N(R^{XN})₂, —C(═O)N(R^{XN})₂, —N(R^{XN})C(═O)Rx“, —SR^{XS}, —S(═O)R^{XS}, —S(═O)₂R^{XS}, —SO₂OR^{XO}, —SO₂N(R^{XN})₂, —CN, and —NO₂; and wherein each R^{XO}, R^{XN} and R^{XS} is independently selected from hydrogen, C₁₋₄alkyl and C₁₋₄haloalkyl.

A compound according to paragraph [0176] wherein each R^{X} is independently selected from halo, —OR^{XO}, —NR^{XN1}R^{XN2}—CN, and —NO₂, wherein each R^{XO}, R^{XN} and R^{XS} are independently selected from hydrogen, C₁₋₄alkyl and C₁₋₄haloalkyl.

A compound according to paragraph [0176] wherein each R^{X} is independently selected from halo, —C₁₋₄alkyl, —CN, and —NO₂.

A compound according to paragraph [0176] wherein each R^{X} is independently selected from —Cl, —CN, —OMe and -Me.

A compound according to paragraph [0176] wherein each R^{X} is independently selected from —CN and —OMe.

A compound according to paragraph [0176] wherein each R^{X} is independently —CN.

A compound according to paragraph [0176] wherein each R^{X} is independently aryl, optionally substituted with one or more substituents R^{XX}, wherein R^{XX} is selected from halo, C₁₋₄alkyl, C₁₋₄haloalkyl, aryl, —OR^{XO}, —C(═O)OR^{XO}, —N(R^{XN})₂, —C(═O)N(R^{XN})₂, —N(R^{XN})C(═O)R^{XN}, —SR^{XS}, —S(═O)R^{XS}, —S(═O)₂R^{XS}, —SO₂OR^{XO}, —SO₂N(R^{XN})₂, —CN, and —NO₂; and wherein each R^{XO}, R^{XN} and R^{XS} is independently selected from hydrogen, C₁₋₄alkyl and C₁₋₄haloalkyl.

A compound according to paragraph [0182] wherein R^{X} is selected from C₆₋₂₀carboaryl and C₅₋₁₂heteroaryl.

A compound according to paragraph [0183] wherein R^{X} is selected from phenyl, naphthyl, furanyl, thienyl, pyrrolyl, imidazolyl, pyrazolyl, triazolyl, tetrazolyl, oxazolyl, isoxazolyl, thiazolyl, isothiazolyl, pyridyl, pyrimidinyl, pyridazinyl, indolyl, isoindolyl, benzofuranyl, isobenzofuranyl, benzothienyl, isobenzothienyl, indazolyl, benzimidazolyl, benzothiazolyl, benzoisothiazolyl, benzoxazolyl, benzoisoxazolyl, quinolinyl, isoquinolinyl, cinnolinyl, or quinazolinyl.

A compound according to paragraph [0183] wherein R^{X} is phenyl.

A compound according to paragraph [0183] wherein R^{X} is C₅₋₆heteroaryl.

A compound according to paragraph [0185] wherein R^{X} is selected from furanyl, thienyl, pyrrolyl, imidazolyl, pyrazolyl, triazolyl, tetrazolyl, oxazolyl, isoxazolyl, thiazolyl, isothiazolyl, pyridyl, pyrimidinyl, or pyridazinyl.

A compound according to any one of claims [0182] to [0187] wherein R^{X} is unsubstituted.

A compound according to any one of claims [0182] to [0187] wherein R^{X} is substituted with one or more substituents R^{XX}, wherein R^{XX} is selected from halo, C₁₋₄alkyl, C₁₋₄haloalkyl, aryl, —OR^{XO}, —C(═O)OR^{XO}, —N(R^{XN})₂, —C(═O)N(R^{XN})₂, —N(R^{XN})C(═O)R^{XN}, —SR^{XS}, —S(═O)R^{XS}, —S(═O)₂R^{XS}, —SO₂OR^{XO}, —SO₂N(R^{XN})₂, —CN, and —NO₂; and wherein each R^{XO}, R^{XN} and R^{XS} is independently selected from hydrogen, C₁₋₄alkyl and C₁₋₄haloalkyl.

A compound according to paragraph [0189] wherein R^{XX} is selected from halo, C₁₋₄alkyl, and C₁₋₄haloalkyl.

A compound according to paragraph [0190] wherein R^{XX} is halo.

A compound according to paragraph [0191] wherein R^{XX} is selected from —F, —Cl, and —Br.

A compound according to paragraph [0191] wherein R^{XX} is —F.

### Certain Preferred Embodiments

In some preferred embodiments, the compound may be a compound of formula (III):

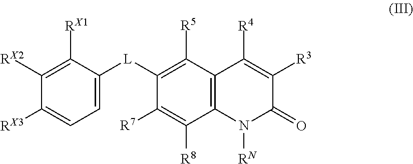

wherein R^{X1}, R^{X2} and R^{X3} are each independent selected from hydrogen and R^{X}.

A compound of formula (III) wherein:
R^{X1} is R^{X}
R^{X2} and R^{X3} are both hydrogen, and
R³, R⁴, R⁵, R⁷, R⁸, R^{X}, L and R^{N} are as defined in any one of paragraphs [0001] to [0192].

A compound of formula (III) wherein:
R^{X2} is R^{X},
R^{X1} and R^{X3} are both hydrogen, and
R³, R⁴, R⁵, R⁷, R⁸, R^{X}, L and R^{N} are as defined in any one of paragraphs [0001] to [0192].

A compound of formula (III) wherein:
R^{X3} is R^{X},
R^{X1} and R^{X2} are both hydrogen, and
R³, R⁴, R⁵, R⁷, R⁸, R^{X}, L and R^{N} are as defined in any one of paragraphs [0001] to [0192].

A compound of formula (III) wherein:
R^{X1} and R^{X3} are each independently R^{X},
R^{X2} is hydrogen, and
R³, R⁴, R⁵, R⁷, R⁸, R^{X}, L and R^{N} are as defined in any one of paragraphs [0001] to [0192].

### Optional Provisos

In some embodiments, the compound is a compound according to any of the preceding paragraphs, with the proviso that the compound is not N-(1,3-dimethyl-2-oxo-1,2-dihydroquinolin-6-yl)-2-methoxybenzenesulfonamide (Compound P-001) (CAS Registry Number 1425927-10-1).

In some embodiments, the compound is a compound according to any of the preceding paragraphs, with the proviso that the compound is not N-(1-methyl-2-oxo-1,2-dihydroquinolin-6-yl)-4-methylbenzenesulfonamide (CAS Registry Number 198639-71-3).

In some embodiments, the compound is a compound according to any of the preceding paragraphs, with the proviso that the compound is not N-(1-methyl-2-oxo-1,2-dihydroquinolin-6-yl)-N′-(methyl)-4-methylbenzenesulfonamide (CAS Registry Number 198639-72-4).

In some embodiments, the compound is a compound according to any of the preceding paragraphs, with the proviso that the compound is not 4-cyano-N-(1,3-dimethyl-2-oxo-1,2-dihydroquinolin-6-yl)-2-methoxybenzenesulfonamide.

### Specific Compounds of the Invention

In some embodiments, the compound is a compound selected from the compounds set out in any of the tables below, or pharmaceutically acceptable salts thereof:

TABLE 1
Substituted N-methylquinolone 6-arylsulfonamides

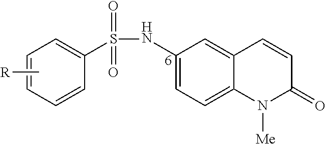

| Compound Ref | R |
|---|---|
| 1 | H |
| 2 | 2-CN |
| 3 | 3-CN |
| 4 | 4-CN |
| 5 | 4-NH₂ |
| 6 | 2-F, 4-CN |
| 7 | 2-Me, 4-CN |
| 8 | 3-Cl, 4-CN |
| 9 | 3-Cl, 4-Cl |
| 39 | 4-NO₂ |
| 40 | 2-Br, 4-CN |

TABLE 2
7-Substituted N-methylquinolone 6-arylsulfonamides

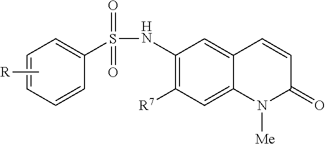

| Compound Ref | R | R⁷ |
|---|---|---|
| 10 | H | MeO |
| 11 | 4-CN | MeO |
| 12 | 4-CN | F |
| 13 | 4-CN | EtO |
| 14 | 4-CN | iPrO |
| 41 | 2-Me, 4-Br | MeO |

TABLE 3
5-Substituted N-methylquinolone 6-arylsulfonamides

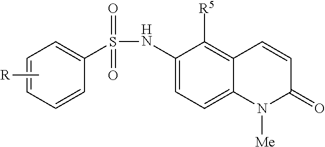

| Compound Ref | R | R⁵ |
|---|---|---|
| 15 | H | C≡CH |
| 16 | 4-CN | MeO |
| 17 | 4-CN | F |
| 18 | 4-CN | Br |
| 42 | 4-Ph | Br |
| 43 | 4-(4-F—Ph) | Br |

TABLE 4
3-Substituted N-methylquinolone 6-arylsulfonamides

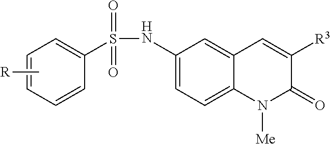

| Compound Ref | R | R³ |
|---|---|---|
| 19 | 4-CN | Me |
| 44 | 4-CN | Et |
| 45 | 2-MeO, 4-CN | Et |
| 62 | 2-MeO, 4-CONH₂ | Me |
| 63 | 4-NH₂ | Me |
| 64 | 4-PhO | Me |
| 65 | 3-NH₂ | Me |
| 66 | 4-NO₂ | Me |

TABLE 5
3,7-Disubstituted N-methylquinolone 6-arylsulfonamides

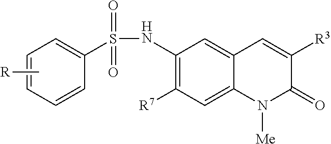

| Compound Ref | R | R³ | R⁷ |
|---|---|---|---|
| 21 | H | Me | MeO |
| 22 | 4-CN | Me | MeO |
| 23 | 2-Me; 4-CN | Me | MeO |
| 24 | 4-NO₂ | Me | MeO |
| 25 | 4-NH₂ | Me | MeO |
| 26 | 2-OEt; 4-CN | Me | MeO |
| 27 | 2-OMe; 4-CN | Me | MeO |
| 28 | 2-OMe | Me | MeO |
| 29 | 4-Me | Me | MeO |
| 67 | 2-OCF₃ | Me | MeO |

TABLE 6
4-Substituted N-methylquinolone 6-arylsulfonamides

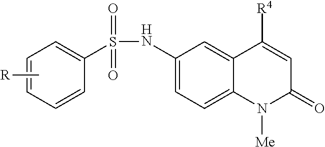

| Compound Ref | R | R⁴ |
|---|---|---|
| 30 | 4-CN | Me |
| 31 | 2-MeO; 4-CN | Me |
| 32 | 4-CN | CF₃ |
| 46 | 2-CN | Me |
| 47 | 3-CN | Me |

TABLE 7
4,7-Disubstituted N-methylquinolone 6-arylsulfonamides

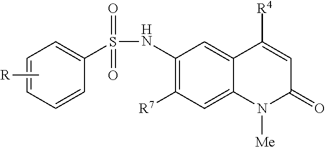

| Compound Ref | R | R⁴ | R⁷ |
|---|---|---|---|
| 33 | 4-CN | Me | MeO |

TABLE 8
3,8-Disubstituted N-methylquinolone 6-arylsulfonamides

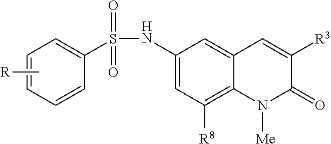

| Compound Ref | R | R³ | R⁸ |
|---|---|---|---|
| 48 | 4-CN | Me | F |
| 49 | 2-MeO, 4-CN | Me | F |

TABLE 9
3,4,7-Trisubstituted N-methylquinolone 6-arylsulfonamides

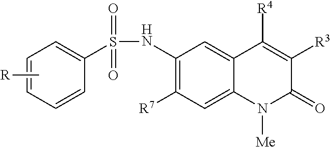

| Compound Ref | R | R³ | R⁴ | R⁷ |
|---|---|---|---|---|
| 50 | 4-CN | Me | Me | MeO |

TABLE 10
N-Methylquinolone 6-alkylsulfonamides

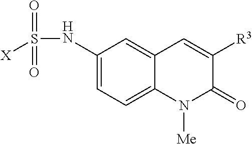

| Compound Ref | X | R³ |
|---|---|---|
| 51 | Me | H |
| 52 | Et | H |
| 53 | iPr | H |
| 54 | c-Pr | H |
| 55 | c-Hex | H |
| 56 | c-Hex | Me |

TABLE 11
N-methylquinolone 6-heteroarylsulfonamides.

| Compound | |
|---|---|
| Ref | Structure |
| 36 | 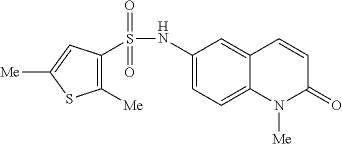 [SMILES: Cc1cc(S(=O)(=O)Nc2ccc3c(ccc(=O)n3C)c2)c(C)s1] |
| 37 | 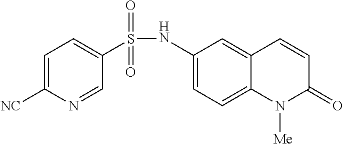 [SMILES: Cn1c(=O)ccc2cc(NS(=O)(=O)c3ccc(C#N)nc3)ccc21] |
| 57 | 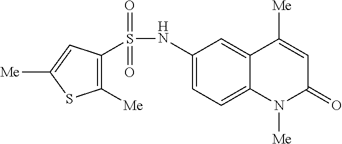 [SMILES: Cc1cc(S(=O)(=O)Nc2ccc3c(c2)c(C)cc(=O)n3C)c(C)s1] |

TABLE 12
N-ethylquinolone 6-arylsulfonamides.

| Compound | |
|---|---|
| Ref | Structure |
| 35 | 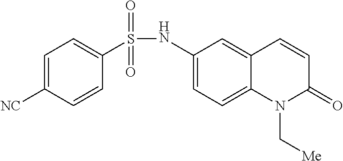 [SMILES: CCn1c(=O)ccc2cc(NS(=O)(=O)c3ccc(C#N)cc3)ccc21] |
| 58 | 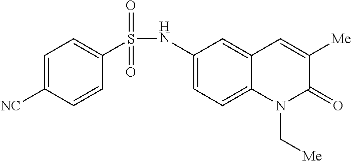 [SMILES: CCn1c(=O)c(C)cc2cc(NS(=O)(=O)c3ccc(C#N)cc3)ccc21] |

TABLE 13
Alternative N-alkylquinolones (N-alkylsulfonamides).

| Compound | |
|---|---|
| Ref | Structure |
| 34 | 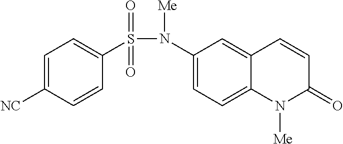 [SMILES: CN(c1ccc2c(ccc(=O)n2C)c1)S(=O)(=O)c1ccc(C#N)cc1] |
| 59 | 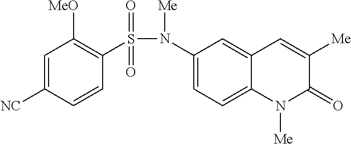 [SMILES: COc1cc(C#N)ccc1S(=O)(=O)N(C)c1ccc2c(c1)cc(C)c(=O)n2C] |
| 68 | 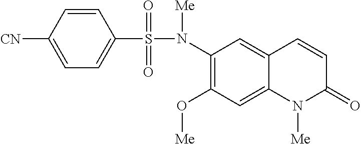 |

TABLE 14
Alternative N-alkylquinolones (reverse sulfonamides).

| Compound | |
|---|---|
| Ref | Structure |
| 38 | 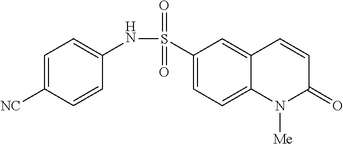 [SMILES: Cn1c(=O)ccc2cc(S(=O)(=O)Nc3ccc(C#N)cc3)ccc21] |
| 60 | 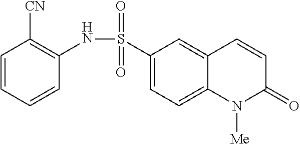 [SMILES: Cn1c(=O)ccc2cc(S(=O)(=O)Nc3ccccc3C#N)ccc21] |
| 61 | 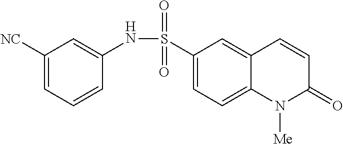 [SMILES: Cn1c(=O)ccc2cc(S(=O)(=O)Nc3cccc(C#N)c3)ccc21] |

In some preferred embodiments, the compound is selected from the following:

| Compound | | |
|---|---|---|
| Ref | Structure | IUPAC name |
| 2 | 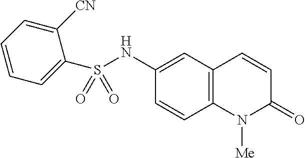 [SMILES: Cn1c(=O)ccc2cc(NS(=O)(=O)c3ccccc3C#N)ccc21] | 2-cyano-N-(1-methyl-2-oxo-6- quinolyl)benzenesulfonamide |
| 3 | 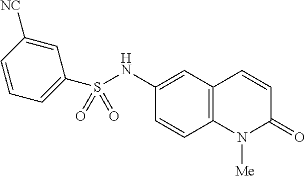 [SMILES: Cn1c(=O)ccc2cc(NS(=O)(=O)c3cccc(C#N)c3)ccc21] | 3-cyano-N-(1-methyl-2-oxo-6- quinolyl)benzenesulfonamide |
| 4 | 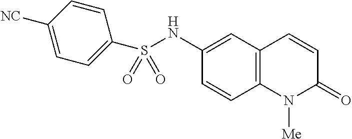 [SMILES: Cn1c(=O)ccc2cc(NS(=O)(=O)c3ccc(C#N)cc3)ccc21] | 4-cyano-N-(1-methyl-2-oxo-6- quinolyl)benzenesulfonamide |
| 9 | 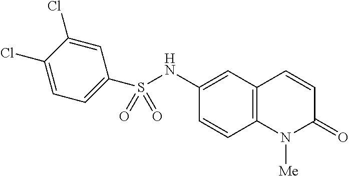 [SMILES: Cn1c(=O)ccc2cc(NS(=O)(=O)c3ccc(Cl)c(Cl)c3)ccc21] | 3,4-dichloro-N-(1-methyl-2-oxo-6- quinolyl)benzenesulfonamide |
| 10 | 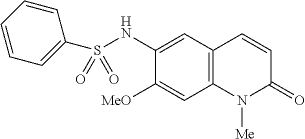 [SMILES: COc1cc2c(ccc(=O)n2C)cc1NS(=O)(=O)c1ccccc1] | N-(7-methoxy-1-methyl-2-oxo-6- quinolyl)benzenesulfonamide |
| 11 | 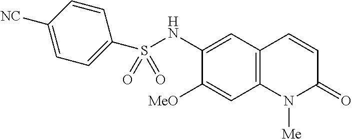 [SMILES: COc1cc2c(ccc(=O)n2C)cc1NS(=O)(=O)c1ccc(C#N)cc1] | 4-cyano-N-(7-methoxy-1-methyl- 2-oxo-6- quinolyl)benzenesulfonamide |
| 16 | 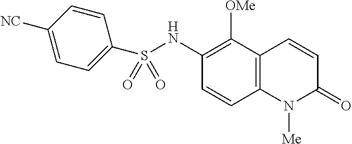 [SMILES: COc1c(NS(=O)(=O)c2ccc(C#N)cc2)ccc2c1ccc(=O)n2C] | 4-cyano-N-(5-methoxy-1-methyl- 2-oxo-6- quinolyl)benzenesulfonamide |
| 18 | 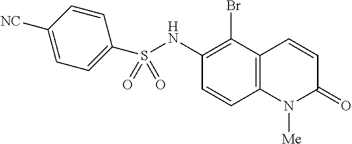 [SMILES: Cn1c(=O)ccc2c(Br)c(NS(=O)(=O)c3ccc(C#N)cc3)ccc21] | 4-cyano-N-(5-bromo-1-methyl-2- oxo-6- quinolyl)benzenesulfonamide |
| 19 | 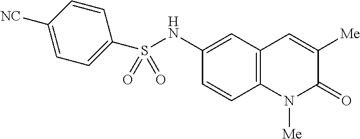 [SMILES: Cc1cc2cc(NS(=O)(=O)c3ccc(C#N)cc3)ccc2n(C)c1=O] | 4-cyano-N-(1,3-dimethyl-2-oxo-6- quinolyl)benzenesulfonamide |
| 21 | 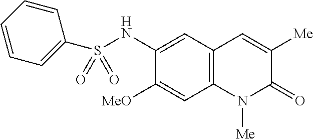 [SMILES: COc1cc2c(cc1NS(=O)(=O)c1ccccc1)cc(C)c(=O)n2C] | N-(1,3-dimethyl-7-methoxy-2-oxo- 6-quinolyl)benzenesulfonamide |
| 22 | 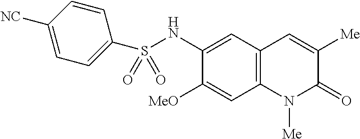 [SMILES: COc1cc2c(cc1NS(=O)(=O)c1ccc(C#N)cc1)cc(C)c(=O)n2C] | 4-cyano-N-(1,3-dimethyl-7- methoxy-2-oxo-6-quinolyl) benzenesulfonamide |
| 30 | 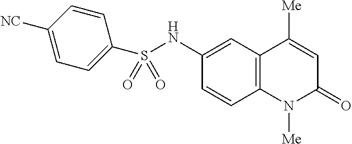 [SMILES: Cc1cc(=O)n(C)c2ccc(NS(=O)(=O)c3ccc(C#N)cc3)cc12] | 4-cyano-N-(1,4-dimethyl-2-oxo-6- quinolyl) benzenesulfonamide |
| 33 | 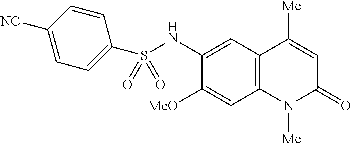 [SMILES: COc1cc2c(cc1NS(=O)(=O)c1ccc(C#N)cc1)c(C)cc(=O)n2C] | 4-cyano-N-(1,4-dimethyl-7- methoxy-2-oxo-6-quinolyl) benzenesulfonamide |
| 34 | 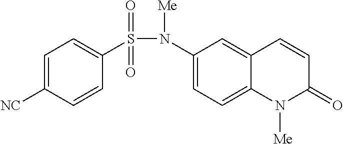 [SMILES: CN(c1ccc2c(ccc(=O)n2C)c1)S(=O)(=O)c1ccc(C#N)cc1] | 4-cyano-N-methyl-N-(1-methyl-2- oxo-6- quinolyl)benzenesulfonamide |
| 35 | 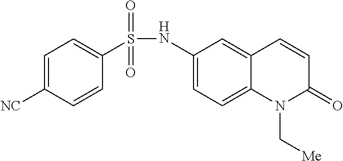 [SMILES: CCn1c(=O)ccc2cc(NS(=O)(=O)c3ccc(C#N)cc3)ccc21] | 4-cyano-N-(1-ethyl-2-oxo-6- quinolyl)benzenesulfonamide |
| 38 | 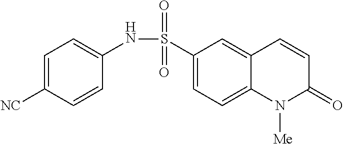 [SMILES: Cn1c(=O)ccc2cc(S(=O)(=O)Nc3ccc(C#N)cc3)ccc21] | N-(4-cyanophenyl)-1-methyl-2- oxo-quinoline-6-sulfonamide |
| 45 | 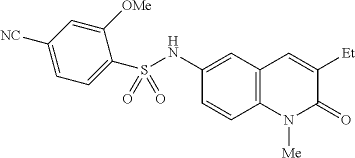 [SMILES: CCc1cc2cc(NS(=O)(=O)c3ccc(C#N)cc3OC)ccc2n(C)c1=O] | 4-cyano-N-(3-ethyl-1-methyl-2- oxo-6-quinolyl)-2-methoxy- benzenesulfonamide |
| 49 | 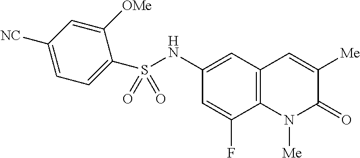 [SMILES: COc1cc(C#N)ccc1S(=O)(=O)Nc1cc(F)c2c(c1)cc(C)c(=O)n2C] | 4-cyano-N-(8-fluoro-1,3-dimethyl- 2-oxo-6-quinolyl)-2-methoxy- benzenesulfonamide |
| 50 | 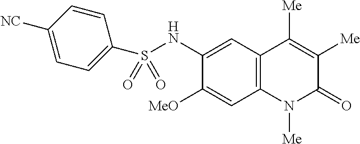 [SMILES: COc1cc2c(cc1NS(=O)(=O)c1ccc(C#N)cc1)c(C)c(C)c(=O)n2C] | 4-cyano-N-(7-methoxy-1,3,4- trimethyl-2-oxo-6- quinolyl)benzenesulfonamide |
| 51 | 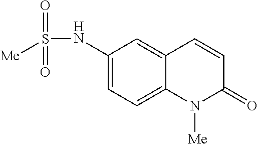 [SMILES: Cn1c(=O)ccc2cc(NS(C)(=O)=O)ccc21] | N-(1-methyl-2-oxo-6- quinolyl)methanesulfonamide |
| 52 | 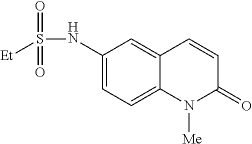 [SMILES: CCS(=O)(=O)Nc1ccc2c(ccc(=O)n2C)c1] | N-(1-methyl-2-oxo-6- quinolyl)ethanesulfonamide |
| 53 | 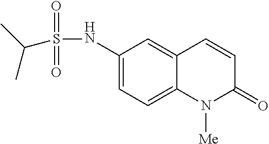 [SMILES: CC(C)S(=O)(=O)Nc1ccc2c(ccc(=O)n2C)c1] | N-(1-methyl-2-oxo-6- quinolyl)propane-2-sulfonamide |
| 54 | 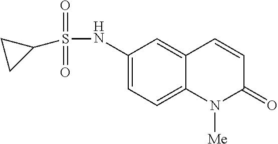 [SMILES: Cn1c(=O)ccc2cc(NS(=O)(=O)C3CC3)ccc21] | N-(1-methyl-2-oxo-6- quinolyl)cyclopropanesulfonamide |
| 55 | 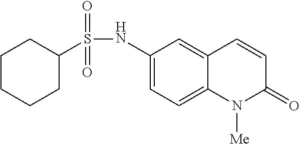 [SMILES: Cn1c(=O)ccc2cc(NS(=O)(=O)C3CCCCC3)ccc21] | N-(1-methyl-2-oxo-6-quinolyl) cyclohexanesulfonamide |
| 56 | 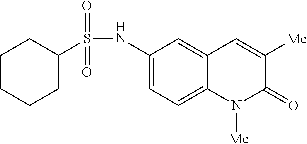 [SMILES: Cc1cc2cc(NS(=O)(=O)C3CCCCC3)ccc2n(C)c1=O] | N-(1,3-dimethyl-2-oxo-6- quinolyl)cyclohexanesulfonamide |
| 59 | 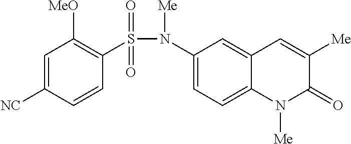 [SMILES: COc1cc(C#N)ccc1S(=O)(=O)N(C)c1ccc2c(c1)cc(C)c(=O)n2C] | 4-cyano-N-(1,3-dimethyl-2-oxo-6- quinolyl)-2-methoxy- N-methyl-benzenesulfonamide |

### Molecular Weight

In some embodiments the compound has a molecular weight of from 300 to 1000.

In some embodiments the bottom of range is from 300, 310, 320, 330, 340, 350, 375, or 400.

In some embodiments, the top of range is 1000, 900, 700, 600, 550 or 500.

In some embodiments, the range is 340 to 550.

### Combinations

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

All combinations of the embodiments pertaining to the chemical groups represented by the variables (e.g., L, X, R^{N}, R³, R⁴, R⁷, R⁸ etc) are specifically embraced by the present invention and are disclosed herein just as if each and every combination was individually and explicitly disclosed, to the extent that such combinations embrace compounds that are stable compounds (i.e., compounds that can be isolated, characterised, and tested for biological activity). In addition, all sub-combinations of the chemical groups listed in the embodiments describing such variables are also specifically embraced by the present invention and are disclosed herein just as if each and every such sub-combination of chemical groups was individually and explicitly disclosed herein.

### Substantially Purified Forms

One aspect of the present invention pertains to compounds as described herein, in substantially purified form and/or in a form substantially free from contaminants.

In one embodiment, the compound is in a substantially purified form with a purity of least 50% by weight, e.g., at least 60% by weight, e.g., at least 70% by weight, e.g., at least 80% by weight, e.g., at least 90% by weight, e.g., at least 95% by weight, e.g., at least 97% by weight, e.g., at least 98% by weight, e.g., at least 99% by weight.

Unless specified, the substantially purified form refers to the compound in any stereoisomeric or enantiomeric form. For example, in one embodiment, the substantially purified form refers to a mixture of stereoisomers, i.e., purified with respect to other compounds. In one embodiment, the substantially purified form refers to one stereoisomer, e.g., optically pure stereoisomer. In one embodiment, the substantially purified form refers to a mixture of enantiomers. In one embodiment, the substantially purified form refers to an equimolar mixture of enantiomers (i.e., a racemic mixture, a racemate). In one embodiment, the substantially purified form refers to one enantiomer, e.g., optically pure enantiomer.

In one embodiment, the compound is in a form substantially free from contaminants wherein the contaminants represent no more than 50% by weight, e.g., no more than 40% by weight, e.g., no more than 30% by weight, e.g., no more than 20% by weight, e.g., no more than 10% by weight, e.g., no more than 5% by weight, e.g., no more than 3% by weight, e.g., no more than 2% by weight, e.g., no more than 1% by weight.

Unless specified, the contaminants refer to other compounds, that is, other than stereoisomers or enantiomers. In one embodiment, the contaminants refer to other compounds and other stereoisomers. In one embodiment, the contaminants refer to other compounds and the other enantiomer.

In some embodiments, the compound is in a substantially purified form with an optical purity of at least 60% (i.e., 60% of the compound, on a molar basis, is the desired enantiomer, and 40% is the undesired enantiomer), e.g., at least 70%, e.g., at least 80%, e.g., at least 90%, e.g., at least 95%, e.g., at least 97%, e.g., at least 98%, e.g., at least 99%.

### Isomers

Certain compounds may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and I-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers,” as used herein, are structural (or constitutional) isomers (i.e., isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hydroxyazo, and nitro/aci-nitro.

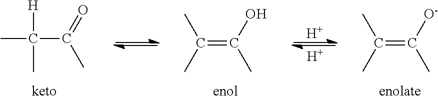

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including mixtures (e.g., racemic mixtures) thereof. Methods for the preparation (e.g., asymmetric synthesis) and separation (e.g., fractional crystallisation and chromatographic means) of such isomeric forms are either known in the art or are readily obtained by adapting the methods taught herein, or known methods, in a known manner.

### Salts

It may be convenient or desirable to prepare, purify, and/or handle a corresponding salt of the compound, for example, a pharmaceutically-acceptable salt. Examples of pharmaceutically acceptable salts are discussed in Berge et al., 1977, “Pharmaceutically Acceptable Salts,” J. Pharm. Sci., Vol. 66, pp. 1-19.

For example, if the compound is anionic, or has a functional group which may be anionic (e.g., —COOH may be —COO⁻), then a salt may be formed with a suitable cation.

Examples of suitable inorganic cations include, but are not limited to, alkali metal ions such as Na⁺ and K⁺, alkaline earth cations such as Ca²⁺ and Mg²⁺, and other cations such as Al⁺³. Examples of suitable organic cations include, but are not limited to, ammonium ion (i.e., NH₄⁺ and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

Unless otherwise specified, a reference to a particular compound also includes salt forms thereof.

### Hydrates and Solvates

It may be convenient or desirable to prepare, purify, and/or handle a corresponding hydrate or solvate of the compound (e.g., pharmaceutically acceptable hydrates or solvates of the compound). The term “solvate” is used herein in the conventional sense to refer to a complex of solute (e.g., compound, salt of compound) and solvent. If the solvent is water, the solvate may be conveniently referred to as a hydrate, for example, a mono-hydrate, a di-hydrate, a tri-hydrate, etc.

Unless otherwise specified, a reference to a particular compound also includes hydrate and solvate forms thereof.

### Chemically Protected Forms

It may be convenient or desirable to prepare, purify, and/or handle the compound in a chemically protected form. The term “chemically protected form” is used herein in the conventional chemical sense and pertains to a compound in which one or more reactive functional groups are protected from undesirable chemical reactions under specified conditions (e.g., pH, temperature, radiation, solvent, and the like). In practice, well known chemical methods are employed to reversibly render unreactive a functional group, which otherwise would be reactive, under specified conditions. In a chemically protected form, one or more reactive functional groups are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts; 4th Edition; John Wiley and Sons, 2006).

A wide variety of such “protecting,” “blocking,” or “masking” methods are widely used and well known in organic synthesis. For example, a compound which has two nonequivalent reactive functional groups, both of which would be reactive under specified conditions, may be derivatized to render one of the functional groups “protected,” and therefore unreactive, under the specified conditions; so protected, the compound may be used as a reactant which has effectively only one reactive functional group. After the desired reaction (involving the other functional group) is complete, the protected group may be “deprotected” to return it to its original functionality.

For example, a hydroxy group may be protected as an ether (—OR) or an ester (—OC(═O)R), for example, as: a t-butyl ether; a benzyl, benzhydryl (diphenylmethyl), or trityl (triphenylmethyl) ether; a trimethylsilyl or t-butyldimethylsilyl ether; or an acetyl ester (—OC(═O)CH₃, —OAc).

For example, an aldehyde or ketone group may be protected as an acetal (R—CH(OR)₂) or ketal (R₂C(OR)₂), respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide (—NRCO—R) or a urethane (—NRCO—OR), for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH—Fmoc), as a 6-nitroveratryloxy amide (—NH—Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulfonyl)ethyloxy amide (—NH—Psec); or, in suitable cases (e.g., cyclic amines), as a nitroxide radical (>N—O.).

For example, a carboxylic acid group may be protected as an ester for example, as: an C₁₋₇alkyl ester (e.g., a methyl ester; a t-butyl ester); a C₁₋₇ haloalkyl ester (e.g., a C₁₋₇trihaloalkyl ester); a triC₁₋₇ alkylsilyl-C₁₋₇alkyl ester; or a C₅₋₂₀ aryl-C₁₋₇ alkyl ester (e.g., a benzyl ester; a nitrobenzyl ester); or as an amide, for example, as a methyl amide. For example, a thiol group may be protected as a thioether (—SR), for example, as: a benzyl thioether; an acetamidomethyl ether (—S—CH₂NHC(═O)CH₃).

For example, a carbonyl group may be protected as an oxime (—C(═NOH)—) or a substituted oxime (—C(═NOR)—), for example, where R is saturated aliphatic C₁₋₄alkyl.

### Prodrugs

It may be convenient or desirable to prepare, purify, and/or handle the compound in the form of a prodrug. The term “prodrug,” as used herein, pertains to a compound which, when metabolised (e.g., in vivo), yields the desired active compound. Typically, the prodrug is inactive, or less active than the desired active compound, but may provide advantageous handling, administration, or metabolic properties.

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Also, some prodrugs are activated enzymatically to yield the active compound, or a compound which, upon further chemical reaction, yields the active compound (for example, as in ADEPT, GDEPT, LIDEPT, etc.). For example, the prodrug may be a sugar derivative or other glycoside conjugate, or may be an amino acid ester derivative.

### Chemical Synthesis

Methods for the chemical synthesis of the compounds of the present invention are described herein. These and/or other well-known methods may be modified and/or adapted in known ways in order to facilitate the synthesis of additional compounds within the scope of the present invention.

In one approach, compounds of the invention may be prepared by condensing an appropriate sulfonyl chloride onto an appropriate 6-aminoquinolone derivative, in the presence of a base.

For example, a compound of the invention of formula (IIa):

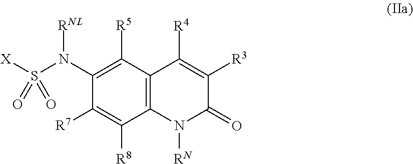

can be prepared from a corresponding 6-amino compound of formula (IV):

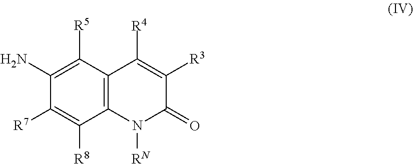

by treatment with a suitable sulfonyl chloride, for example of formula X—SO₂—Cl. In some embodiments, the compound of formula (IV) and the sulfonyl chloride are mixed together in the presence of a base, such as pyridine.

Compounds of formula (IV) can be prepared by methods known in the art. For example, a compound of formula (IV) may be prepared by reduction of the corresponding 6-nitro compound:

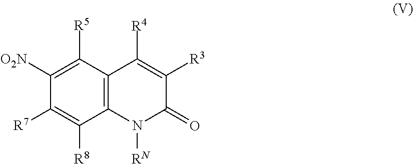

In some embodiments, reduction comprises treatment with a reducing agent. For example, reduction methods include, but are not limited to, treatment with tin(II) chloride and hydrochloric acid, or treatment with iron powder and ammonium chloride. Other suitable methods are known in the art.

Nitro compounds of formula (V) can be prepared, for example, by nitration of the corresponding quinolone compounds:

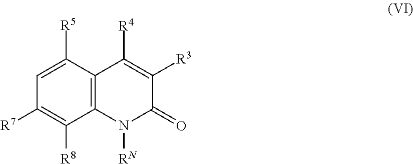

Nitration may be performed by methods known in the art including, but not limited to, treatment with a nitrating agent such as concentrated nitric acid or potassium nitrate, and concentrated sulfuric acid.

The corresponding quinolone compounds (VI) are commercially available or can be prepared by methods known in the art.

In an alternative approach, the 6-amino intermediates of formula (IV) may be prepared from the corresponding 6-halo (preferably 6-bromo) compounds, for example a compound of formula (VII):

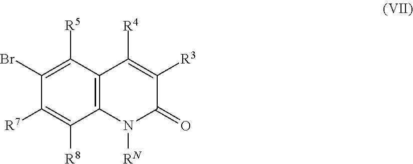

Conversion of the 6-bromo compounds to the corresponding amino compound (IV) may be effected, for example, by treatment with ammonium hydroxide in the presence of a copper catalyst (e.g. Cu₂O).

The 6-bromo compounds (VII) can be prepared, for example, by bromination of the corresponding quinolone compounds (VI).

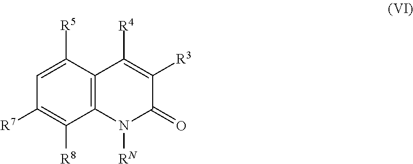

Bromination may be performed by methods known in the art including, but not limited to, treatment with a brominating agent, such as N-bromosuccinimide (NBS).

The corresponding quinolone compounds (VI) are commercially available or can be prepared by methods known in the art.

In other embodiments, 6-bromo compounds of formula (VII) may be prepared directly, by cyclisation of a precursor compound e.g. of formula (VIII) or (IX):

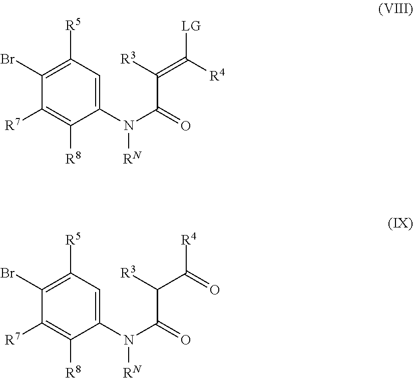

wherein LG is a leaving group, for example an alkoxy group, such as —OEt.

In further embodiments, a compound of the invention of formula (IIb):

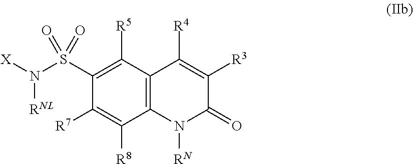

can be prepared, for example, from a corresponding 6-sufonyl halide (e.g. a sulfonyl chloride) of formula (X):

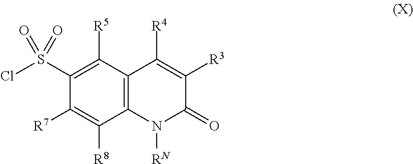

by reaction with a suitable amine, for example a compound of formula X—NH₂. In some embodiments, the compound of formula (X) and the amine are mixed together in the presence of a base, such as dimethylaminopyridine (DMAP).

Compounds of the invention wherein R^{NL} is other than hydrogen may be prepared, for example, by alkylation of the corresponding unsubstituted sulfonamide. For example, an N-alkyl sulfonamide may be prepared by treatment with a base (e.g. NaH) and an alkyl halide (e.g. MeI).

Variations of the above-described synthetic methods, and alternative synthetic methods, would be evident to the skilled person in view of the description herein and the examples provided below.

### Compositions

One aspect of the present invention pertains to a composition (e.g., a pharmaceutical composition) comprising a compound of the invention, as described herein, and a pharmaceutically acceptable carrier, diluent, or excipient.

Another aspect of the present invention pertains to a method of preparing a composition (e.g., a pharmaceutical composition) comprising admixing a compound of the invention, as described herein, and a pharmaceutically acceptable carrier, diluent, or excipient.

### Uses

The compounds of the invention described herein are useful, for example, in the treatment of proliferative disorders, such as, for example, cancer, etc.

### Use in Methods of Therapy

Another aspect of the present invention pertains to a compound of the invention, as described herein, for use in a method of treatment of the human or animal body by therapy.

### Use in the Manufacture of Medicaments

Another aspect of the present invention pertains to use of a compound of the invention, as described herein, in the manufacture of a medicament for use in treatment.

In one embodiment, the medicament comprises the compound.

### Methods of Treatment

Another aspect of the present invention pertains to a method of treatment comprising administering to a patient in need of treatment a therapeutically effective amount of a compound of the invention, as described herein, preferably in the form of a pharmaceutical composition.

### Conditions Treated

In some embodiments (e.g., of use in methods of therapy, of use in the manufacture of medicaments, of methods of treatment), treatment is characterised by modulation of the BRPF1/HOX pathway.

In some embodiments, treatment is characterised by modulation of HOX gene expression/loss.

In some embodiments, treatment is characterised by modulation of BRPF complex formation with at least one lysine acyl transferase selected from MOZ, MORF and HBO1.

### Conditions Treated—Proliferative Disorders and Cancer

In some embodiments (e.g., of use in methods of therapy, of use in the manufacture of medicaments, of methods of treatment), the treatment is treatment of a proliferative disorder.

The term “proliferative condition,” as used herein, pertains to an unwanted or uncontrolled cellular proliferation of excessive or abnormal cells which is undesired, such as, neoplastic or hyperplastic growth.

In some embodiments, the treatment is treatment of: a proliferative condition characterised by benign, pre-malignant, or malignant cellular proliferation, including but not limited to tumours and cancers (see below).

In some embodiments, the treatment is treatment of cancer.

In some embodiments, the cancer is characterised by activation of the BRPF1/HOX pathway.

Examples of cancers include, but are not limited to, adrenal cancer, anal cancer, bladder cancer, bone cancer, bowel cancer, brain/CNS tumours, breast cancer, cervical cancer, endometrial cancer, esophagus cancer, eye cancer, gallbladder cancer, Hodgkin disease, Kaposi sarcoma, kidney cancer, leukemia (such, for example, acute myeloid leukemia (AML), acute lymphocytic leukemia (ALL), chronic lymphocytic leukemia (CLL), chromic myeloid leukemia (CML), chronic myelomonocytic leukemia (CMML)), liver cancer, lung cancer (such as, for example small cell and non-small cell lung cancer), lymphoma, malignant mesothelioma, multiple myeloma, myelodysplastic syndrome, neuroblastoma, non-Hodgkin lymphoma, osteosarcoma, ovarian cancer, pancreatic cancer, pituitary tumours, prostate cancer, retinoblastoma, rhabdomyosarcoma, sarcoma, skin cancer (such as, for example, basal and squamous cell skin cancer, melanoma, merkel cell skin cancer), stomach cancer, testicular cancer, thyroid cancer, uterine cancer.

In some embodiments, the treatment is treatment of lung cancer.

In particular embodiments, the treatment is treatment of small cell lung cancer.

In some embodiments, the treatment is treatment of leukemia.

In particular embodiments, the treatment is treatment of acute myeloid leukemia (AML).

An anti-cancer effect may arise through one or more mechanisms, including but not limited to, the regulation of cell proliferation, the inhibition of cell cycle progression, the inhibition of angiogenesis (the formation of new blood vessels), the inhibition of metastasis (the spread of a tumour from its origin), the inhibition of invasion (the spread of tumour cells into neighbouring normal structures), or the promotion of apoptosis (programmed cell death). The compounds of the present invention may be used in the treatment of the cancers described herein, independent of the mechanisms discussed herein.

### Conditions Treated—Other Disorders

In some embodiments (e.g., of use in methods of therapy, of use in the manufacture of medicaments, of methods of treatment), the treatment is treatment of a disorder other than cancer, such as, for example, a pulmonary disorder, an inflammatory disorder, a neurological disorder, or fibrosis.

In some embodiments, the treatment is treatment of a pulmonary disorder. In particular embodiments, the treatment is treatment of primary pulmonary hypertension or emphysema.

In some embodiments, the treatment is treatment of a neurological disorder.

In some embodiments, the treatment is treatment of a neurological disorder associated with abnormal expression of MOZ or MORF genes, for example DiGeorge syndrome, Noonan syndrome-like disorder, Ohdo syndrome, genitopatellar syndrome, blepharophimosis-ptosis-epicanthus inversus syndrome.

### Treatment

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, alleviation of symptoms of the condition, amelioration of the condition, and cure of the condition. Treatment as a prophylactic measure (i.e., prophylaxis) is also included. For example, use with patients who have not yet developed the condition, but who are at risk of developing the condition, is encompassed by the term “treatment.”

For example, treatment includes the prophylaxis of cancer, reducing the incidence of cancer, alleviating the symptoms of cancer, etc.

The term “therapeutically-effective amount,” as used herein, pertains to that amount of a compound, or a material, composition or dosage form comprising a compound, which is effective for producing some desired therapeutic effect, commensurate with a reasonable benefit/risk ratio, when administered in accordance with a desired treatment regimen.

### Combination Therapies

The term “treatment” includes combination treatments and therapies, in which two or more treatments or therapies are combined, for example, sequentially or simultaneously. For example, the compounds described herein may also be used in combination therapies, e.g., in conjunction with other agents, for example, cytotoxic agents, anticancer agents, molecularly-targeted agents, etc. Examples of treatments and therapies include, but are not limited to, chemotherapy (the administration of active agents, including, e.g., drugs, antibodies (e.g., as in immunotherapy), prodrugs (e.g., as in photodynamic therapy, GDEPT, ADEPT, etc.); surgery; radiation therapy; photodynamic therapy; gene therapy; and controlled diets.

For example, it may be beneficial to combine treatment with a compound of the invention as described herein with one or more other (e.g., 1, 2, 3, 4) agents or therapies that regulates cell growth or survival or differentiation via a different mechanism, thus treating several characteristic features of cancer development.

One aspect of the present invention pertains to a compound of the invention as described herein, in combination with one or more additional therapeutic agents, as described below.

The particular combination would be at the discretion of the physician who would select dosages using his common general knowledge and dosing regimens known to a skilled practitioner.

The agents (i.e., the compound of the invention described herein, plus one or more other agents) may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g., 1, 2, 3, 4 or more hours apart, or even longer periods apart where required), the precise dosage regimen being commensurate with the properties of the therapeutic agent(s).

The agents (i.e., the compound of the invention described here, plus one or more other agents) may be formulated together in a single dosage form, or alternatively, the individual agents may be formulated separately and presented together in the form of a kit, optionally with instructions for their use.

### Other Uses

The compounds of the invention described herein may also be used as cell culture additives to inhibit cell proliferation, etc.

The compounds of the invention described herein may also be used as part of an in vitro assay, for example, in order to determine whether a candidate host is likely to benefit from treatment with the compound in question.

The compounds of the invention described herein may also be used as a standard, for example, in an assay, in order to identify other compounds, other anti-proliferative agents, other anti-cancer agents, etc.

### Kits

One aspect of the invention pertains to a kit comprising (a) a compound of the invention as described herein, or a composition comprising a compound of the invention as described herein, e.g., preferably provided in a suitable container and/or with suitable packaging; and (b) instructions for use, e.g., written instructions on how to administer the compound or composition.

The written instructions may also include a list of indications for which the active ingredient is a suitable treatment.

### Routes of Administration

The compound of the invention or pharmaceutical composition comprising the compound of the invention may be administered to a subject by any convenient route of administration, whether systemically/peripherally or topically (i.e., at the site of desired action).

Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### The Subject/Patient

The subject/patient may be a chordate, a vertebrate, a mammal, a placental mammal, a marsupial (e.g., kangaroo, wombat), a monotreme (e.g. a platypus), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human.

Furthermore, the subject/patient may be any of its forms of development, for example, a foetus.

In one preferred embodiment, the subject/patient is a human.

### Formulations

While it is possible for the compound of the invention to be administered alone, it is preferable to present it as a pharmaceutical formulation (e.g., composition, preparation, medicament) comprising at least one compound of the invention, as described herein, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents. The formulation may further comprise other active agents, for example, other therapeutic or prophylactic agents.

Thus, the present invention further provides pharmaceutical compositions, as defined above, and methods of making a pharmaceutical composition comprising admixing at least one compound of the invention, as described herein, together with one or more other pharmaceutically acceptable ingredients well known to those skilled in the art, e.g., carriers, diluents, excipients, etc. If formulated as discrete units (e.g., tablets, etc.), each unit contains a predetermined amount (dosage) of the compound.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

Suitable carriers, diluents, excipients, etc. can be found in standard pharmaceutical texts, for example, Remington's Pharmaceutical Sciences, 18th edition, Mack Publishing Company, Easton, Pa., 1990; and Handbook of Pharmaceutical Excipients, 5th edition, 2005.

The formulations may be prepared by any methods well known in the art of pharmacy. Such methods include the step of bringing into association the compound with a carrier which constitutes one or more accessory ingredients. In general, the formulations are prepared by uniformly and intimately bringing into association the compound with carriers (e.g., liquid carriers, finely divided solid carrier, etc.), and then shaping the product, if necessary.

The formulation may be prepared to provide for rapid or slow release; immediate, delayed, timed, or sustained release; or a combination thereof.

Formulations may suitably be in the form of liquids, solutions (e.g., aqueous, non-aqueous), suspensions (e.g., aqueous, non-aqueous), emulsions (e.g., oil-in-water, water-in-oil), elixirs, syrups, electuaries, mouthwashes, drops, tablets (including, e.g., coated tablets), granules, powders, losenges, pastilles, capsules (including, e.g., hard and soft gelatin capsules), cachets, pills, ampoules, boluses, suppositories, pessaries, tinctures, gels, pastes, ointments, creams, lotions, oils, foams, sprays, mists, or aerosols.

Formulations may suitably be provided as a patch, adhesive plaster, bandage, dressing, or the like which is impregnated with one or more compounds and optionally one or more other pharmaceutically acceptable ingredients, including, for example, penetration, permeation, and absorption enhancers. Formulations may also suitably be provided in the form of a depot or reservoir.

The compound may be dissolved in, suspended in, or admixed with one or more other pharmaceutically acceptable ingredients. The compound may be presented in a liposome or other microparticulate which is designed to target the compound, for example, to blood components or one or more organs.

Formulations suitable for oral administration (e.g., by ingestion) include liquids, solutions (e.g., aqueous, non-aqueous), suspensions (e.g., aqueous, non-aqueous), emulsions (e.g., oil-in-water, water-in-oil), elixirs, syrups, electuaries, tablets, granules, powders, capsules, cachets, pills, ampoules, boluses.

Formulations suitable for buccal administration include mouthwashes, losenges, pastilles, as well as patches, adhesive plasters, depots, and reservoirs. Losenges typically comprise the compound in a flavored basis, usually sucrose and acacia or tragacanth. Pastilles typically comprise the compound in an inert matrix, such as gelatin and glycerin, or sucrose and acacia. Mouthwashes typically comprise the compound in a suitable liquid carrier.

Formulations suitable for sublingual administration include tablets, losenges, pastilles, capsules, and pills.

Formulations suitable for oral transmucosal administration include liquids, solutions (e.g., aqueous, non-aqueous), suspensions (e.g., aqueous, non-aqueous), emulsions (e.g., oil-in-water, water-in-oil), mouthwashes, losenges, pastilles, as well as patches, adhesive plasters, depots, and reservoirs.

Formulations suitable for non-oral transmucosal administration include liquids, solutions (e.g., aqueous, non-aqueous), suspensions (e.g., aqueous, non-aqueous), emulsions (e.g., oil-in-water, water-in-oil), suppositories, pessaries, gels, pastes, ointments, creams, lotions, oils, as well as patches, adhesive plasters, depots, and reservoirs. Formulations suitable for transdermal administration include gels, pastes, ointments, creams, lotions, and oils, as well as patches, adhesive plasters, bandages, dressings, depots, and reservoirs.

Tablets may be made by conventional means, e.g., compression or moulding, optionally with one or more accessory ingredients. Compressed tablets may be prepared by compressing in a suitable machine the compound in a free-flowing form such as a powder or granules, optionally mixed with one or more binders (e.g., povidone, gelatin, acacia, sorbitol, tragacanth, hydroxypropylmethyl cellulose); fillers or diluents (e.g., lactose, microcrystalline cellulose, calcium hydrogen phosphate); lubricants (e.g., magnesium stearate, talc, silica); disintegrants (e.g., sodium starch glycolate, cross-linked povidone, cross-linked sodium carboxymethyl cellulose); surface-active or dispersing or wetting agents (e.g., sodium lauryl sulfate); preservatives (e.g., methyl p-hydroxybenzoate, propyl p-hydroxybenzoate, sorbic acid); flavours, flavour enhancing agents, and sweeteners. Moulded tablets may be made by moulding in a suitable machine a mixture of the powdered compound moistened with an inert liquid diluent. The tablets may optionally be coated or scored and may be formulated so as to provide slow or controlled release of the compound therein using, for example, hydroxypropylmethyl cellulose in varying proportions to provide the desired release profile. Tablets may optionally be provided with a coating, for example, to affect release, for example an enteric coating, to provide release in parts of the gut other than the stomach.

Ointments are typically prepared from the compound and a paraffinic or a water-miscible ointment base.

Creams are typically prepared from the compound and an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the compound through the skin or other affected areas. Examples of such dermal penetration enhancers include dimethylsulfoxide and related analogues.

Emulsions are typically prepared from the compound and an oily phase, which may optionally comprise merely an emulsifier (otherwise known as an emulgent), or it may comprises a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulgents and emulsion stabilisers include Tween 60, Span 80, cetostearyl alcohol, myristyl alcohol, glyceryl monostearate and sodium lauryl sulfate. The choice of suitable oils or fats for the formulation is based on achieving the desired cosmetic properties, since the solubility of the compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for intranasal administration, where the carrier is a liquid, include, for example, nasal spray, nasal drops, or by aerosol administration by nebuliser, include aqueous or oily solutions of the compound.

Formulations suitable for intranasal administration, where the carrier is a solid, include, for example, those presented as a coarse powder having a particle size, for example, in the range of about 20 to about 500 microns which is administered in the manner in which snuff is taken, i.e., by rapid inhalation through the nasal passage from a container of the powder held close up to the nose.

Formulations suitable for pulmonary administration (e.g., by inhalation or insufflation therapy) include those presented as an aerosol spray from a pressurised pack, with the use of a suitable propellant, such as dichlorodifluoromethane, trichlorofluoromethane, dichloro-tetrafluoroethane, carbon dioxide, or other suitable gases.

Formulations suitable for ocular administration include eye drops wherein the compound is dissolved or suspended in a suitable carrier, especially an aqueous solvent for the compound.

Formulations suitable for rectal administration may be presented as a suppository with a suitable base comprising, for example, natural or hardened oils, waxes, fats, semi-liquid or liquid polyols, for example, cocoa butter or a salicylate; or as a solution or suspension for treatment by enema.

Formulations suitable for vaginal administration may be presented as pessaries, tampons, creams, gels, pastes, foams or spray formulations containing in addition to the compound, such carriers as are known in the art to be appropriate.

Formulations suitable for parenteral administration (e.g., by injection), include aqueous or non-aqueous, isotonic, pyrogen-free, sterile liquids (e.g., solutions, suspensions), in which the compound is dissolved, suspended, or otherwise provided (e.g., in a liposome or other microparticulate). Such liquids may additional contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Typically, the concentration of the compound in the liquid is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets.

### Dosage

It will be appreciated by one of skill in the art that appropriate dosages of the compounds of the invention, and compositions comprising the compounds of the invention, can vary from patient to patient. Determining the optimal dosage will generally involve the balancing of the level of therapeutic benefit against any risk or deleterious side effects. The selected dosage level will depend on a variety of factors including, but not limited to, the activity of the particular compound of the invention, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of the compound of the invention is in the range of about 10 μg to about 250 mg (more typically about 100 μg to about 25 mg) per kilogram body weight of the subject per day. Where the compound is a salt, an ester, an amide, a prodrug, or the like, the amount administered is calculated on the basis of the parent compound and so the actual weight to be used is increased proportionately.

### EXAMPLES

The following examples are provided solely to illustrate the present invention and are not intended to limit the scope of the invention, as described herein.

### Synthesis Example 1

### Preparation of 6-amino-1-methylquinolin-2(1H)-one

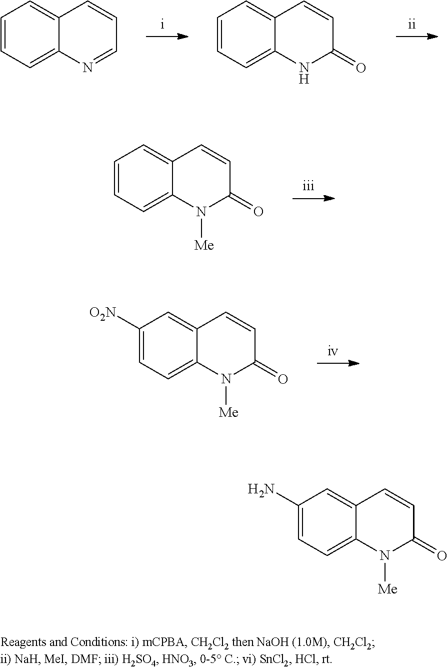 [SMILES: c1ccc2ncccc2c1 ; O=c1ccc2ccccc2[nH]1 ; Cn1c(=O)ccc2ccccc21 ; Cn1c(=O)ccc2cc([N+](=O)[O-])ccc21 ; Cn1c(=O)ccc2cc(N)ccc21]

STEP 1: To a stirred solution of quinoline (1.00 g, 7.75 mmol, 1 eq.) in DCM (10 mL) at 0° C. was added 3-chloroperbenzoic acid (77%, 1.74 g, 1 eq.) portionwise over 10 minutes. The resulting solution was allowed to warm to room temperature and then stirred overnight. After completion of the reaction the solution was washed with sodium hydroxide (1.0 M, 30 mL) and the aqueous phase extracted with DCM (3×40 mL). The organic layers were combined and dried over anhydrous MgSO₄, filtered and the solvent removed in vacuo to yield quinolone-N-oxide (1.02 g, 6.98 mmol, 91%) which was used in the next step without further purification.

To a stirred solution of quinolone-N-oxide (1.00 g, 6.97 mmol) in DCM (15 mL) was added sodium hydroxide (1.0 M, 10 mL) and the resulting biphasic mixture was cooled to 0° C. To this was added, under rapid agitation, benzoyl chloride (1.18 g, 0.97 mL, 8.37 mmol, 1.2 eq) dropwise. The suspension was stirred for 2 hours and the resulting precipitate was collected by filtration, washed with water (50 mL) and dried under vacuum to give quinolin-2(1H)-one (0.88 g, 6.13 mmol, 78% over 2 steps). ¹H NMR (400 MHz, DMSO-d₆): δ ppm 11.75 (1H, br, s), 7.90 (1H, d, J=9.5 Hz), 7.65 (1H, dd, J=7.8, 1.6 Hz), 7.49 (1H, t, J=8.1 Hz), 7.35-7.26 (1H, m), 7.22-7.09 (1H, m), 6.50 (1H, d, J=9.5 Hz).

STEP 2: To a solution of quinolin-2(1H)-one (500 mg, 3.51 mmol, 1 eq.) in dry DMF (5 mL) under an argon atmosphere was added NaH (60%, 168 mg, 4.23 mmol, 1.2 eq.). Upon the completion of gas evolution, iodomethane (602 mg, 264 μL, 4.23 mmol, 1.2 eq) was added in 1 portion and the resulting solution was stirred overnight. Excess sodium hydride was quenched by the addition of water (4 mL) and the solvents were removed in vacuo. The residue was dissolved in ethyl acetate (15 mL), washed with water and then brine. The organic phase was dried over anhydrous MgSO₄, filtered and then concentrated in vacuo. The crude solid was purified by column chromatography (3:2 ethyl acetate:hexanes) to give 1-methylquinolin-2(1H)-one (382 mg, 2.51 mmol, 69%) as a pale yellow solid. ¹H NMR (400 MHz, DMSO-d₆): δ ppm 7.91 (1H, d, J=9.5 Hz), 7.72 (1H, dd, J=7.7, 1.5 Hz), 7.67-7.61 (1H, m), 7.53 (1H, d, J=8.5 Hz), 7.30-7.25 (1H, m), 6.62 (1H, d, J=9.5 Hz), 3.62 (3H, s).

STEP 3: To a suspension of 1-methylquinolin-2(1H)-one (1.2 g, 7.55 mmol, 1 eq.) in concentrated H₂SO₄ (10 mL) at −5° C. was added HNO₃ (70%, 3 mL) dropwise. The resulting yellow solution was stirred at this temperature for 2.5 hours before being allowed to warm to room temperature. The solution was next poured over crushed ice and the resultant suspension stirred for 5 minutes. The precipitate was collected by filtration and dried under vacuum to give 1-methyl-6-nitroquinolin-2(1H)-one (1.34 g, 6.57 mmol, 87%) as a yellow solid. ¹H NMR (400 MHz, DMSO-d₆): δ ppm 8.75 (1H, d, J=2.7 Hz), 8.41 (1H, dd, J=9.4, 2.7 Hz), 8.16 (1H, d, J=9.5 Hz), 7.73 (1H, d, J=9.4 Hz), 6.81 (1H, d, J=9.5 Hz), 3.68 (3H, s).

STEP 4: To a suspension of 1-methyl-6-nitroquinolin-2(1H)-one (180 mg, 0.88 mmol, 1 eq.) in concentrated HCl (5 mL) was added SnCl₂.2H₂O (1.0 g, 4.41 mmol, 5 eq.) and the resulting suspension was stirred overnight. Sodium hydroxide was added until all solids had dissolved and the solution had turned bright yellow (˜pH 10). The aqueous solution was then extracted with DCM (3×100 mL) and the organic layers were combined and the solvent removed in vacuo to give 6-amino-1-methylquinolin-2(1H)-one (147 mg, 0.85 mmol, 97%). ¹H NMR (400 MHz, CDCl₃): δ ppm 7.45 (1H, d, J=9.5 Hz), 7.13 (1H, d, J=8.9 Hz), 6.90 (1H, dd, J=8.9, 2.7 Hz), 6.76 (1H, d, J=2.7 Hz), 6.60 (1H, d, J=9.5 Hz), 3.66-3.58 (5H, m).

### General Procedure 1: Conversion of 6-amino-1-methylquinolin-2(1H)-one to arylsulfonamide derivatives

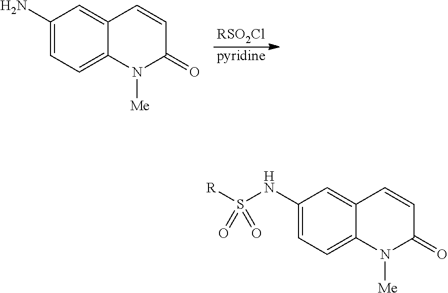

To a solution of aniline derivative in DCM (0.1 M) was added pyridine (2 eq.) followed by the appropriate sulfonyl chloride (1.5 eq.). The reaction was stirred overnight and then diluted with acetone until a homogenous solution was achieved. The solution was concentrated onto Celite® and purified by column chromatography (acetone:hexanes) to yield the desired arylsulfonamide derivative.

### N-(1-methyl-2-oxo-1,2-dihydroquinolin-6-yl)benzenesulfonamide (Compound 1)

¹H NMR (400 MHz, DMSO-d₆): δ ppm 10.32 (1H, br. s), 7.83 (1H, d, J=9.6 Hz), 7.76 (1H, s), 7.64-7.50 (3H, m), 7.46-7.39 (2H, m), 7.31 (1H, dd, J=10.4, 2.0 Hz), 6.58 (1H, d, J=9.4 Hz), 3.54 (3H, s).

### 2-cyano-N-(1-methyl-2-oxo-1,2-dihydroquinolin-6-yl)benzenesulfonamide (Compound 2)

¹H NMR (400 MHz, DMSO-d₆): δ ppm 8.19 (1H, t, J=1.5 Hz), 8.15-8.07 (1H, m), 8.04-7.96 (1H, m), 7.87 (1H, d, J=9.5 Hz), 7.81-7.71 (1H, m), 7.50-7.40 (2H, m), 7.31 (1H, s), 6.60 (1H, d, J=9.5 Hz), 3.54 (3H, s).

### 3-cyano-N-(1-methyl-2-oxo-1,2-dihydroquinolin-6-yl)benzenesulfonamide (Compound 3)

¹H NMR (400 MHz, DMSO-d₆): δ ppm 10.53 (1H, br. s), 8.19 (1H, t, J=1.5 Hz), 8.11 (1H, dt, J=7.8, 1.3 Hz), 8.03-7.97 (1H, m), 7.87 (1H, d, J=9.5 Hz), 7.80-7.72 (1H, m), 7.48-7.41 (2H, m), 7.33-7.26 (1H, m), 6.63-6.56 (1H, m), 3.55 (3H, s).

### 4-cyano-N-(1-methyl-2-oxo-1,2-dihydroquinolin-6-yl)benzenesulfonamide (Compound 4)

¹H NMR (400 MHz, DMSO-d₆): δ ppm 10.59 (1H, br. s), 8.06-8.01 (2H, m), 7.91-7.84 (3H, m), 7.47-7.42 (2H, m), 7.30 (1H, d, J=2.6 Hz), 6.60 (1H, d, J=9.5 Hz), 3.55 (3H, s).

### 4-amino-N-(1-methyl-2-oxo-1,2-dihydroquinolin-6-yl)benzenesulfonamide (Compound 5)

¹H NMR (400 MHz, DMSO-d₆): δ ppm 10.32 (1H, br. s), 7.85 (1H, d, J=9.6 Hz), 7.76 (1H, s), 7.36 (2H, d, J=8.7 Hz) 7.31 (1H, dd, J=10.4, 2.0 Hz), 6.55-6.44 (3H, m), 3.54 (3H, s).

### 4-cyano-2-fluoro-N-(1-methyl-2-oxo-1,2-dihydroquinolin-6-yl)benzenesulfonamide (Compound 6)

¹H NMR (500 MHz, DMSO-d₆) δ ppm 8.10 (1H, d, J=10.1 Hz), 7.95 (1H, t, J=7.7 Hz), 7.84 (2H, t, J=9.5 Hz), 7.47-7.39 (2H, m), 7.33 (1H, dd, J=9.1, 2.5 Hz), 6.58 (1H, d, J=9.5 Hz), 3.55 (3H, s).

### 4-cyano-2-methyl-N-(1-methyl-2-oxo-1,2-dihydroquinolin-6-yl)benzenesulfonamide (Compound 7)

¹H NMR (400 MHz, DMSO-d₆) δ ppm 7.96 (1H, d, J=8.3 Hz), 7.90 (1H, s), 7.80 (2H, t, J=9.2 Hz), 7.47-7.19 (3H, m), 6.57 (1H, d, J=9.1 Hz), 3.53 (3H, s), 2.64 (3H, s).

### 3-chloro-4-cyano-N-(1-methyl-2-oxo-1,2-dihydroquinolin-6-yl)benzenesulfonamide (Compound 8)

¹H NMR (400 MHz, DMSO-d₆) δ ppm 8.16 (1H, d, J=8.1 Hz), 8.03 (1H, d, J=1.5 Hz), 7.88 (1H, d, J=9.6 Hz), 7.79 (1H, dd, J=8.2, 1.6 Hz), 7.49-7.42 (2H, m), 7.31 (1H, dd, J=9.1, 2.5 Hz), 6.61 (1H, d, J=9.3 Hz), 3.56 (3H, s).

### 3,4-dichloro-N-(1-methyl-2-oxo-1,2-dihydroquinolin-6-yl)benzenesulfonamide (Compound 9)

¹H NMR (400 MHz, DMSO-d₆): δ ppm 10.62-10.32 (1H, br. s), 7.95 (1H, d, J=2.2 Hz), 7.88 (1H, d, J=9.5 Hz), 7.83 (1H, d, J=8.5 Hz), 7.65 (1H, dd, J=8.5, 2.2 Hz), 7.45 (2H, m, J=2.0 Hz), 7.31 (1H, dd, J=9.0, 2.6 Hz), 6.60 (1H, d, J=9.5 Hz), 3.55 (3H, s).

### 2,5-dimethyl-N-(1-methyl-2-oxo-1,2-dihydroquinolin-6-yl)thiophene-3-sulfonamide (Compound 36)

¹H NMR (400 MHz, DMSO-d₆) δ ppm 10.23 (1H, br. s), 7.87 (1H, d, J=9.6 Hz), 7.47 (1H, d, J=9.1 Hz), 7.41 (1H, d, J=2.5 Hz), 7.32 (1H, dd, J=9.1, 2.5 Hz), 6.88 (1H, d, J=1.3 Hz), 6.60 (1H, d, J=9.3 Hz), 3.56 (3H, s), 2.40 (3H, s), 2.31 (3H, s).

### 6-cyano-N-(1-methyl-2-oxo-1,2-dihydroquinolin-6-yl)pyridine-3-sulfonamide (Compound 37)

¹H NMR (400 MHz, DMSO-d₆) δ ppm 10.76 (1H, s), 8.99 (1H, dd, J=2.3, 0.8 Hz), 8.30 (1H, dd, J=8.1, 2.3 Hz), 8.21 (1H, dd, J=8.1, 0.8 Hz), 7.86 (1H, d, J=9.6 Hz), 7.52-7.39 (2H, m), 7.32-7.21 (1H, m), 6.60 (1H, d, J=9.3 Hz), 3.55 (3H, s).

### N-(1-methyl-2-oxo-1,2-dihydroquinolin-6-yl)-4-nitrobenzenesulfonamide (Compound 39)

¹H NMR (400 MHz, DMSO-d₆) δ ppm 8.39-8.32 (2H, m), 8.01-7.94 (2H, m), 7.85 (1H, d, J=9.4 Hz), 7.47-7.41 (2H, m), 7.33-7.26 (1H, m), 6.59 (1H, d, J=9.4 Hz), 3.54 (3H, s).

### 2-bromo-4-cyano-N-(1-methyl-2-oxo-1,2-dihydroquinolin-6-yl)benzenesulfonamide (Compound 40)

¹H NMR (400 MHz, DMSO-d₆) δ ppm 10.65 (1H, s), 8.16 (1H, d, J=8.3 Hz), 8.03 (1H, d, J=1.5 Hz), 7.88 (1H, d, J=9.6 Hz), 7.79 (1H, dd, J=8.2, 1.6 Hz), 7.51-7.41 (2H, m), 7.31 (1H, dd, J=8.8, 2.5 Hz), 6.61 (1H, d, J=9.6 Hz), 3.56 (3H, s).

### N-(1-methyl-2-oxo-1,2-dihydroquinolin-6-yl)methanesulfonamide (Compound 51)

¹H NMR (400 MHz, DMSO-d₆) δ ppm 9.79 (1H, s), 7.91 (1H, d, J=9.3 Hz), 7.58-7.52 (2H, m), 7.46 (1H, dd, J=9.6, 2.5 Hz), 6.63 (1H, d, J=9.3 Hz), 3.60 (3H, s), 2.99 (3H, s).

### N-(1-methyl-2-oxo-1,2-dihydroquinolin-6-yl)ethanesulfonamide (Compound 52)

¹H NMR (400 MHz, DMSO-d₆) δ ppm 9.86 (1H, s), 7.90 (1H, d, J=9.3 Hz), 7.56-7.51 (2H, m), 7.47 (1H, dd, J=10.4, 2.0 Hz), 6.62 (1H, d, J=9.3 Hz), 3.60 (3H, s), 3.08 (2H, q, J=7.3 Hz), 1.21 (3H, t, J=7.3 Hz).

### N-(1-methyl-2-oxo-1,2-dihydroquinolin-6-yl)propane-2-sulfonamide (Compound 53)

¹H NMR (400 MHz, DMSO-d₆) δ ppm 9.85 (1H, s), 7.90 (1H, d, J=9.6 Hz), 7.57-7.45 (3H, m), 6.61 (1H, d, J=9.3 Hz), 3.59 (3H, s), 3.22 (1H, quin, J=6.8 Hz), 1.25 (6H, d, J=6.8 Hz).

### N-(1-methyl-2-oxo-1,2-dihydroquinolin-6-yl)cyclopropanesulfonamide (Compound 54)

¹H NMR (500 MHz, DMSO-d₆) δ ppm 9.79 (1H, s), 7.91 (1H, d, J=9.5 Hz), 7.57 (1H, s), 7.54 (1H, d, J=9.5 Hz), 7.49 (1H, d, J=8.5 Hz), 6.63 (1H, d, J=9.5 Hz), 3.61 (3H, s), 0.91 (4H, br. s).

### N-(1-methyl-2-oxo-1,2-dihydroquinolin-6-yl)cyclohexanesulfonamide (Compound 55)

¹H NMR (400 MHz, DMSO-d₆) δ ppm 9.85 (1H, s), 7.90 (1H, d, J=9.6 Hz), 7.56-7.45 (3H, m), 6.61 (1H, d, J=9.6 Hz), 3.59 (3H, s), 3.04-2.82 (1H, m), 2.03 (2H, d, J=11.1 Hz), 1.75 (2H, d, J=12.6 Hz), 1.57 (1H, d, J=11.6 Hz), 1.51-1.32 (2H, m), 1.26-1.02 (3H, m).

The following compound of the invention was synthesised using methods analogous to those set out above with corresponding starting materials.
4-(Trifluoromethyl)quinolin-2(1H)-one (i.e., analogous to the product of step 1 of Synthesis Example 1) was prepared according to van Oeveren.²⁰
4-(Trifluoromethyl)quinolin-2(1H)-one was converted to 6-amino-1-methyl-4-(trifluoromethyl)quinolin-2-one as outlined in steps 2, 3 and 4 of Synthesis Example 1.
6-Amino-1-methyl-4-(trifluoromethyl)quinolin-2-one was converted to Compound 32 using General Procedure 1.

### 4-cyano-N-(1-methyl-2-oxo-4-(trifluoromethyl)-1,2-dihydroquinolin-6-yl)benzenesulfonamide (Compound 32)

¹H NMR (500 MHz, DMSO-d₆) δ ppm 10.77 (1H, s), 8.06 (2H, d, J=8.5 Hz), 7.88 (2H, d, J=8.5 Hz), 7.65 (1H, d, J=9.1 Hz), 7.48 (1H, dd, J=9.1, 1.9 Hz), 7.45 (1H, s), 7.11 (1H, s), 3.61 (3H, s).

### Synthesis Example 2

### 6-amino-5-bromo-1-methylquinolin-2(1H)-one

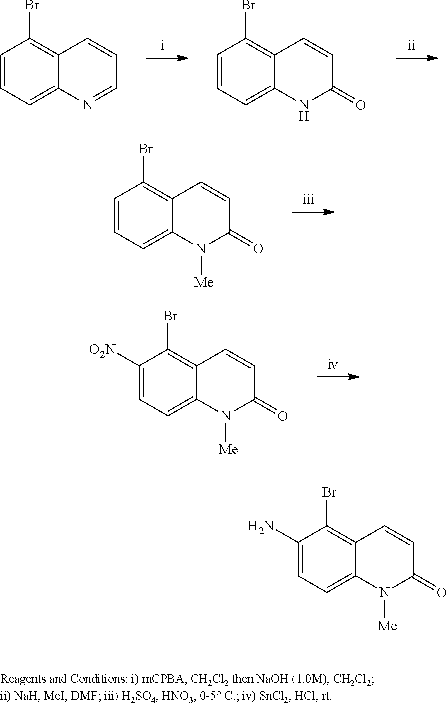 [SMILES: Brc1cccc2ncccc12 ; O=c1ccc2c(Br)cccc2[nH]1 ; Cn1c(=O)ccc2c(Br)cccc21 ; Cn1c(=O)ccc2c(Br)c([N+](=O)[O-])ccc21 ; Cn1c(=O)ccc2c(Br)c(N)ccc21]

STEP 1: 5-bromoquinolin-2(1H)-one was prepared analogously to STEP 1 of Synthesis Example 1. Yield: 69% as a white solid. ¹H NMR (400 MHz, DMSO-d₆): δ ppm 11.98 (1H, br. s), 8.03 (1H, d, J=9.9 Hz), 7.49-7.46 (1H, m), 7.42 (1H, t, J=7.9 Hz), 7.34 (1H, d, J=8.6 Hz), 6.64 (1H, d, J=9.9 Hz).

STEP 2: 5-bromo-1-methylquinolin-2(1H)-one was prepared analogously to STEP 2 of Synthesis Example 1. Yield: 57% as a white solid. ¹H NMR (CDCl₃): δ ppm 8.39 (1H, d, J=8.0 Hz), 7.84 (1H, d, J=8.1 Hz), 7.30 (1H, dd, J=8.1, 7.9 Hz), 7.13 (1H, d, J=7.9 Hz), 6.81 (1H, d, J=7.9 Hz), 3.58 (3H, s).

STEP 3: 5-bromo-1-methyl-6-nitroquinolin-2(1H)-one was prepared analogously to STEP 3 of Synthesis Example 1. Yield: 83% as a yellow solid. ¹H NMR (400 MHz, DMSO-d₆): δ ppm 8.26 (1H, d, J=10.1 Hz), 8.21 (1H, d, J=9.4 Hz), 7.77 (1H, d, J=9.4 Hz), 6.91 (1H, d, J=9.9 Hz), 3.68 (3H, s).

STEP 4: 6-amino-5-bromo-1-methylquinolin-2(1H)-one was prepared analogously to STEP 4 of Synthesis Example 1. Yield: 78% as a bright yellow solid. ¹H NMR (400 MHz, DMSO-d₆): δ ppm 7.98 (1H, d, J=9.9 Hz), 7.39 (1H, d, J=9.1 Hz), 7.18 (1H, d, J=9.1 Hz), 6.66 (1H, d, J=9.9 Hz), 5.39 (2H, br. s), 3.58 (3H, s).

Conversion of 6-amino-5-bromo-1-methylquinolin-2(1H)-one to arylsulfonamide derivatives were carried out as described in General Procedure 1.

### N-(5-bromo-1-methyl-2-oxo-1,2-dihydroquinolin-6-yl)-4-cyanobenzenesulfonamide (Compound 18)

¹H NMR (400 MHz, DMSO-d₆): δ ppm 10.46 (1H, br. s), 8.11-7.96 (3H, m), 7.84 (2H, d, J=8.3 Hz), 7.57 (1H, d, J=9.4 Hz), 7.40 (1H, d, J=9.1 Hz), 6.73 (1H, d, J=9.9 Hz), 3.61 (3H, s).

### N-(5-bromo-1-methyl-2-oxo-1,2-dihydroquinolin-6-yl)-[1,1′-biphenyl]-4-sulfonamide (Compound 42)

¹H NMR (400 MHz, DMSO-d₆) δ ppm 10.14 (1H, s), 8.04 (1H, d, J=9.9 Hz), 7.88 (2H, d, J=8.6 Hz), 7.81-7.71 (4H, m), 7.57 (1H, d, J=9.3 Hz), 7.50 (2H, t, J=7.1 Hz), 7.44 (2H, t, J=9.1 Hz), 6.73 (1H, d, J=9.9 Hz), 3.61 (3H, s).

### N-(5-bromo-1-methyl-2-oxo-1,2-dihydroquinolin-6-yl)-4′-fluoro-[1,1′-biphenyl]-4-sulfonamide (Compound 43)

¹H NMR (400 MHz, DMSO-d₆) δ ppm 10.14 (1H, s), 8.05 (1H, d, J=9.9 Hz), 7.86 (2H, d, J=8.3 Hz), 7.83-7.74 (4H, m), 7.57 (1H, d, J=9.1 Hz), 7.43 (1H, d, J=9.3 Hz), 7.34 (2H, t, J=8.8 Hz), 6.73 (1H, d, J=9.9 Hz), 3.61 (3H, s).

The following compounds of the invention were synthesised using methods analogous to those set out above with corresponding starting materials.

An intermediate step (between step 1 and step 2 of Synthesis Example 2) was used during the synthesis of Compound 15. The intermediate step was a Sonogashira coupling using trimethylsilylacetylene, CuI, and PdCl₂(PPh₃)₂ on 6-amino-5-bromoquinolin-2(1H)-one to give 6-amino-5-ethynylquinolin-2(1H)-one. 6-Amino-5-ethynylquinolin-2(1H)-one was then used in steps 2, 3 and 4 of Synthesis Example 2 to give 6-amino-5-ethynyl-1-methylquinolin-2(1H)-one. 6-Amino-5-ethynyl-1-methylquinolin-2(1H)-one was converted to Compound 15 using General Procedure 1.

### N-(5-ethynyl-1-methyl-2-oxo-1,2-dihydroquinolin-6-yl)benzenesulfonamide (Compound 15)

¹H NMR (400 MHz, CDCl₃) δ ppm 7.96 (1H, d, J=9.6 Hz), 7.89 (1H, d, J=9.1 Hz), 7.81-7.73 (2H, m), 7.58-7.57 (1H, m), 7.47-7.40 (2H, m), 7.35 (1H, d, J=9.1 Hz), 7.18 (1H, br s), 6.76 (1H, d, J=9.9 Hz), 3.70 (3H, s), 3.67 (1H, s).

5-Fluoroquinoline (commercially available) was used as the starting material to obtain Compound 17 using the method outlined in Synthesis Example 2.

### 4-cyano-N-(5-fluoro-1-methyl-2-oxo-1,2-dihydroquinolin-6-yl)benzenesulfonamide (Compound 17)

¹H NMR (400 MHz, DMSO-d₆) δ ppm 10.54 (1H, br. s), 8.06 (2H, d, J=8.6 Hz), 7.92-7.82 (3H, m), 7.45-7.37 (1H, m), 7.33 (1H, d, J=9.6 Hz), 6.66 (1H, d, J=9.6 Hz), 3.58 (3H, s).

### Synthesis Example 3

### 6-amino-1,3-dimethylquinolin-2(1H)-one

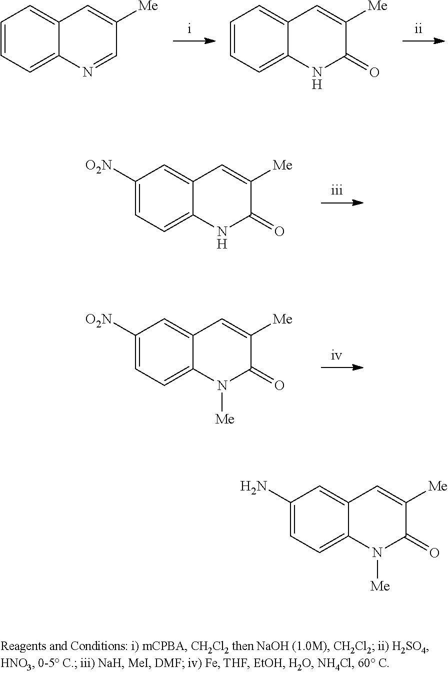 [SMILES: Cc1cnc2ccccc2c1 ; Cc1cc2ccccc2[nH]c1=O ; Cc1cc2cc([N+](=O)[O-])ccc2[nH]c1=O ; Cc1cc2cc([N+](=O)[O-])ccc2n(C)c1=O ; Cc1cc2cc(N)ccc2n(C)c1=O]

STEP 1: 3-methylquinolin-2(1H)-one prepared analogously to Synthesis Example 1. Yield 73%. ¹H NMR (400 MHz, DMSO-d₆): δ ppm 11.72 (1H, br. s.), 7.75 (1H, s), 7.56 (1H, d, J=7.8 Hz), 7.41 (1H, t, J=8.3 Hz), 7.29 (1H, d, J=8.3 Hz), 7.14 (1H, t, J=7.5 Hz), 2.09 (3H, s).

STEPS 2 and 4: were carried out as described in WO2013/027168.³

STEP 3: was carried out analogously to step 3 of Synthesis Example 1.

Conversion of 6-amino-1,3-dimethylquinolin-2(1H)-one to arylsulfonamide derivatives were carried out as described in General Procedure 1.

### 4-cyano-N-(1,3-dimethyl-2-oxo-1,2-dihydroquinolin-6-yl)benzenesulfonamide (Compound 19)

¹H NMR (400 MHz, DMSO-d₆): δ ppm 10.52 (1H, br. s), 8.03 (2H, d, J=8.6 Hz), 7.88 (2H, d, J=8.6 Hz), 7.73 (1H, s), 7.41 (1H, d, J=9.1 Hz), 7.34 (1H, d, J=2.3 Hz), 7.23 (1H, dd, J=9.0, 2.4 Hz), 3.58 (3H, s), 2.10 (3H, s).

### N-(1,3-dimethyl-2-oxo-1,2-dihydroquinolin-6-yl)cyclohexanesulfonamide (Compound 56)

¹H NMR (400 MHz, DMSO-d₆) δ ppm 9.81 (1H, s), 7.78 (1H, s), 7.51-7.26 (3H, m), 3.62 (3H, s), 3.00-2.91 (1H, m), 2.12 (3H, s), 2.02 (2H, d, J=11.4 Hz), 1.74 (2H, d, J=10.6 Hz), 1.57 (1H, d, J=13.9 Hz), 1.41 (2H, m), 1.27-1.04 (3H, m).

### 4-(N-(1,3-dimethyl-2-oxo-1,2-dihydroquinolin-6-yl)sulfamoyl)-3-methoxybenzamide (Compound 62)

¹H NMR (500 MHz, DMSO-d₆) δ ppm 10.12 (1H, s), 8.11 (1H, s), 7.78 (1H, d, J=8.2 Hz), 7.69 (1H, s), 7.62-7.51 (2H, m), 7.45 (1H, dd, J=8.0, 1.4 Hz), 7.36 (1H, d, J=9.1 Hz), 7.31 (1H, d, J=2.5 Hz), 7.27 (1H, dd, J=9.1, 2.2 Hz), 3.97 (3H, s), 3.55 (3H, s), 2.08 (3H, s).

### 4-amino-N-(1,3-dimethyl-2-oxo-1,2-dihydroquinolin-6-yl)benzenesulfonamide (Compound 63)

¹H NMR (400 MHz, DMSO-d₆) δ ppm 8.92 (1H, br. s), 7.69 (1H, s), 7.36 (3H, d, J=8.8 Hz), 7.30-7.21 (2H, m), 6.56-6.46 (2H, m), 5.93 (2H, br. s), 3.57 (3H, s), 2.09 (3H, d, J=1.0 Hz).

### N-(1,3-dimethyl-2-oxo-1,2-dihydroquinolin-6-yl)-4-phenoxybenzenesulfonamide (Compound 64)

¹H NMR (400 MHz, DMSO-d₆) δ ppm 10.27 (1H, br. s), 7.82-7.75 (3H, m), 7.55-7.43 (3H, m), 7.38 (1H, d, J=2.5 Hz), 7.34-7.21 (2H, m), 7.17-7.03 (4H, m), 3.63 (3H, s), 2.20-2.10 (3H, m).

### 3-amino-N-(1,3-dimethyl-2-oxo-1,2-dihydroquinolin-6-yl)benzenesulfonamide (Compound 65)

¹H NMR (400 MHz, DMSO-d₆) δ ppm 10.10 (1H, s), 7.68 (1H, s), 7.38 (1H, d, J=9.1 Hz), 7.32 (1H, d, J=2.5 Hz), 7.29-7.20 (1H, m), 7.12 (1H, t, J=7.8 Hz), 6.93 (1H, t, J=2.0 Hz), 6.88-6.80 (1H, m), 6.69 (1H, ddd, J=8.1, 2.3, 1.0 Hz), 5.53 (2H, s), 3.57 (3H, s), 2.09 (3H, d, J=1.0 Hz).

### N-(1,3-dimethyl-2-oxo-1,2-dihydroquinolin-6-yl)-4-nitrobenzenesulfonamide (Compound 66)

¹H NMR (400 MHz, DMSO-d₆) δ ppm 10.58 (1H, br. S), 8.41-8.30 (2H, m), 8.02-7.87 (2H, m), 7.72 (1H, s), 7.40 (1H, d, J=9.1 Hz), 7.34 (1H, d, J=2.3 Hz), 7.24 (1H, dd, J=8.8, 2.5 Hz), 3.56 (3H, s), 2.06 (3H, s).

### Synthesis Example 4

### Preparation of 6-amino-7-methoxy-1-methylquinolin-2(1H)-one

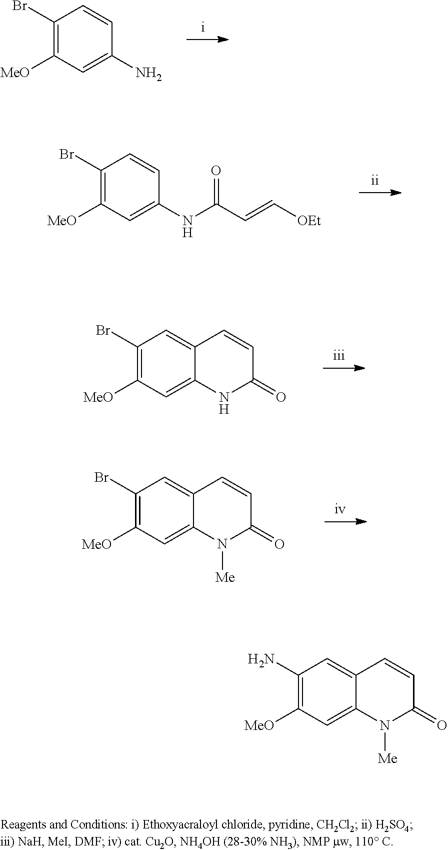 [SMILES: COc1cc(N)ccc1Br ; CCO/C=C/C(=O)Nc1ccc(Br)c(OC)c1 ; COc1cc2[nH]c(=O)ccc2cc1Br ; COc1cc2c(ccc(=O)n2C)cc1Br ; COc1cc2c(ccc(=O)n2C)cc1N]

STEP 1: To a solution of 3-methoxy-4-bromoaniline (1.01 g, 5.00 mmol, 1 eq.) and pyridine (474 mg, 484 μL, 6 mmol, 1.2 eq.) in DCM (20 mL) was added ethoxyacraloyl chloride (in accordance with the method of Fernandez et al)⁴ (432 mg, 6 mmol, 1.2 eq.). The solution was stirred for 3 hours, the solvent removed in vacuo and the residue purified by column chromatography (1:9-6:4; ethyl acetate:hexanes) to give (E)-N-(4-bromo-3-methoxyphenyl)-3-ethoxyacrylamide (885 mg, 2.95 mmol, 59%). ¹H NMR (400 MHz, DMSO-d₆): δ ppm 9.86 (1H, s), 7.55-7.47 (2H, m), 7.44 (1H, d, J=8.6 Hz), 7.11 (1H, dd, J=8.6, 2.3 Hz), 5.51 (1H, d, J=12.1 Hz), 3.96 (2H, q, J=6.9 Hz), 3.83 (3H, s), 1.27 (3H, q, J=7.6 Hz).

STEP 2: Concentrated sulphuric acid (8 mL) was cooled to 0° C. and (E)-N-(4-bromo-3-methoxyphenyl)-3-ethoxyacrylamide (880 mg, 2.93 mmol) was added portionwise. The dark solution was allowed to stir for 20 minutes and was then poured onto ice. The resulting precipitate was filtered, washed with water and dried under vacuum to afford 6-bromo-7-methoxyquinolin-2(1H)-one (745 mg, 2.93 mmol, 100%) as a brown solid ¹H NMR (400 MHz, DMSO-d₆): δ ppm 7.94 (1H, s), 7.80 (1H, d, J=9.6 Hz), 6.94 (1H, s), 6.37 (1H, d, J=9.6 Hz), 3.89 (3H, s).

STEP 3: To a solution of 6-bromo-7-methoxyquinolin-2(1H)-one (200 mg, 0.81 mmol, 1 eq.) in dry DMF (5 mL) under an argon atmosphere was added NaH (60%, 23 mg, 0.97 mmol, 1.2 eq.). Upon the completion of gas evolution, iodomethane (228 mg, 100 μL, 1.71 mmol, 1.3 eq.) was added in 1 portion and the resulting solution was stirred for 3 hours. Excess sodium hydride was quenched by the addition of water (1 mL) and the solvents were removed in vacuo. The residue was dissolved in ethyl acetate (15 mL), washed with water and then brine. The organic phase was dried over anhydrous MgSO₄, filtered and then concentrated in vacuo. Purification by column chromatography (1:4 ethyl acetate:hexanes) gave 6-bromo-7-methoxy-1-methylquinolin-2(1H)-one (132 mg, 0.50 mmol, 56%) as a pale yellow solid. ¹H NMR (400 MHz, DMSO-d₆): δ ppm 8.00 (1H, s), 7.81 (1H, d, J=9.6 Hz), 7.04 (1H, s), 6.49 (1H, d, J=9.6 Hz), 4.02 (3H, s), 3.64 (3H, s).

STEP 4: 6-bromo-7-methoxy-1-methylquinolin-2(1H)-one (200 mg, 0.76 mmol, 1.0 eq.) was dissolved in NMP (1.5 mL) in a Biotage 10 mL microwave vial. Cu₂O (10 mg, 0.076 mmol, 0.1 eq) and NH₄OH (28-30% NH₃, 2 mL) were added and the vial was sealed and heated at 110° C. under microwave irradiation for 5 hours. After cooling to RT the solution was filtered through a pad of Celite® and washed with DCM (20 mL). The filtrate was washed with an aqueous lithium chloride solution (0.5 M, 10 mL) and the organic fractions were combined and concentrated in vacuo. The resulting residue was purified by column chromatography (7:3-3:7 hexanes:acetone) to give 6-amino-7-methoxy-1-methylquinolin-2(1H)-one (41 mg, 0.31 mmol, 35%) as an orange solid. ¹H NMR (400 MHz, DMSO-d₆) δ ppm 7.63 (1H, d, J=9.3 Hz) 6.86 (2H, d, J=11.4 Hz) 6.36 (1H, d, J=9.3 Hz) 4.77 (2H, s) 3.95 (3H, s) 3.60 (3H, s).

Conversion of 6-amino-7-methoxy-1-methylquinolin-2(1H)-one to arylsulfonamide derivatives were carried out as described in General Procedure 1.

### N-(7-methoxy-1-methyl-2-oxo-1,2-dihydroquinolin-6-yl)benzenesulfonamide (Compound 10)

¹H NMR (400 MHz, DMSO-d₆): δ ppm 9.62 (1H, s), 7.83 (1H, d, J=9.4 Hz), 7.69 (2H, d, J=7.3 Hz), 7.64-7.48 (4H, m), 6.83 (1H, s), 6.47-6.42 (1H, m), 3.58 (6H, s).

### 4-cyano-N-(7-methoxy-1-methyl-2-oxo-1,2-dihydroquinolin-6-yl)benzenesulfonamide (Compound 11)

¹H NMR (400 MHz, DMSO-d₆): δ ppm 9.99 (1H, br. s), 8.03 (2H, d, J=8.3 Hz), 7.89-7.80 (3H, m), 7.60 (1H, s), 6.83 (1H, s), 6.46 (1H, d, J=9.6 Hz), 3.58 (3H, s), 3.54 (3H, s).

### 4-cyano-N-(7-fluoro-1-methyl-2-oxo-1,2-dihydroquinolin-6-yl)benzenesulfonamide (Compound 12)

¹H NMR (400 MHz, DMSO-d₆) δ ppm 10.52 (1H, br. s), 8.06 (2H, d, J=8.6 Hz), 7.92 (1H, d, J=9.6 Hz), 7.87 (2H, d, J=8.6 Hz), 7.65 (1H, d, J=8.6 Hz), 7.41 (1H, d, J=12.6 Hz), 6.59 (1H, d, J=9.6 Hz), 3.53 (3H, s).

### 4-cyano-N-(7-ethoxy-1-methyl-2-oxo-1,2-dihydroquinolin-6-yl)benzenesulfonamide (Compound 13)

¹H NMR (500 MHz, DMSO-d₆) δ ppm 9.96 (1H, br. s), 8.03 (2H, d, J=7.9 Hz), 7.87 (1H, d, J=9.5 Hz), 7.81 (2H, d, J=7.9 Hz), 7.63 (1H, s), 6.81 (1H, s), 6.45 (1H, d, J=9.1 Hz), 3.87 (2H, q, J=6.3 Hz), 3.56 (3H, s), 1.06 (3H, t, J=6.8 Hz).

### 4-cyano-N-(7-isopropoxy-1-methyl-2-oxo-1,2-dihydroquinolin-6-yl)benzenesulfonamide (Compound 14)

¹H NMR (400 MHz, DMSO-d₆) δ ppm 9.87 (1H, br. s.), 8.03 (2H, d, J=8.6 Hz), 7.84-7.78 (3H, m), 7.64 (1H, s), 6.80 (1H, s), 6.44 (1H, d, J=9.6 Hz), 4.67 (1H, spt, J=6.0 Hz), 3.55 (3H, s), 1.01 (6H, d, J=6.1 Hz).

### 4-bromo-N-(7-methoxy-1-methyl-2-oxo-1,2-dihydroquinolin-6-yl)-2-methylbenzenesulfonamide (Compound 41)

¹H NMR (500 MHz, DMSO-d₆) δ ppm 9.78 (1H, s), 7.84 (1H, d, J=9.5 Hz), 7.68 (1H, s), 7.59 (1H, s), 7.47 (1H, d, J=8.5 Hz), 7.44 (1H, d, J=8.5 Hz), 6.82 (1H, s), 6.45 (1H, d, J=9.5 Hz), 3.59 (3H, s), 3.57 (3H, s), 2.66 (3H, s).

The following compound of the invention was synthesised using methods analogous to those set out above with corresponding starting materials.

6-Bromo-8-fluoro-3-methylquinolin-2(1H)-one (i.e., analogous to the product of step 3 in Synthesis Example 4) was prepared by adapting the method described in Manimaran et al.²¹ 6-Bromo-8-fluoro-3-methylquinolin-2(1H)-one was converted to 6-amino-8-fluoro-1,3-dimethyl-quinolin-2-one using the procedure outlined in steps 3 and 4 of Synthesis Example 4. 6-Amino-8-fluoro-1,3-dimethyl-quinolin-2-one was converted to Compound 49 using General Procedure 1.

### 4-cyano-N-(8-fluoro-1,3-dimethyl-2-oxo-1,2-dihydroquinolin-6-yl)-2-methoxybenzenesulfonamide (Compound 49)

¹H NMR (500 MHz, DMSO-d₆) δ ppm 10.55 (1H, s), 7.94 (1H, d, J=8.2 Hz), 7.73 (2H, d, J=12.9 Hz), 7.52 (1H, s), 7.14-7.04 (2H, m), 3.96 (3H, s), 3.71 (3H, d, J=8.2 Hz), 2.08 (3H, s).

### Synthesis Example 5

### Preparation of 6-amino-7-methoxy-1-methylquinolin-2(1H)-one

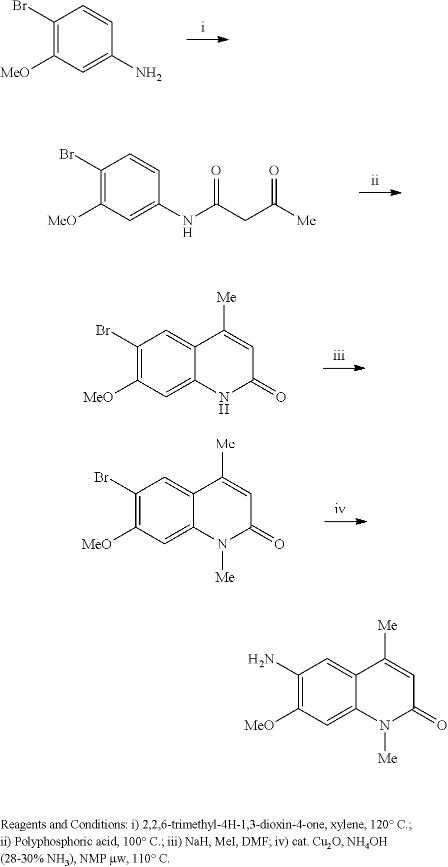 [SMILES: COc1cc(N)ccc1Br ; COc1cc(NC(=O)CC(C)=O)ccc1Br ; COc1cc2[nH]c(=O)cc(C)c2cc1Br ; COc1cc2c(cc1Br)c(C)cc(=O)n2C ; COc1cc2c(cc1N)c(C)cc(=O)n2C]

STEP 1: To a solution of 4-bromo-3-methoxyaniline (2.5 g, 12.32 mmol, 1 eq.) in xylenes (25 mL) at 110° C. was added 2,2,6-Trimethyl-4H-1,3-dioxin-4-one (1.93 g, 1.80 mL, 13.55 mmol, 1.1 eq.). The solution was stirred for 2 hours and allowed to cool to RT. The solvents were removed in vacuo and the residue purified by column chromatography (8:2-6:4; hexanes:ethyl acetate) to yield N-(4-bromo-3-methoxyphenyl)-3-oxobutanamide (2.32 g, 8.14 mmol, 66%) as a brown oil. ¹H NMR (400 MHz, CDCl₃): δ ppm 9.07 (1H, br. s), 7.30 (1H, t, J=2.2 Hz), 7.24 (1H, t, J=8.1 Hz), 7.06 (1H, dd, J=8.0, 1.1 Hz), 6.70 (1H, dd, J=8.2, 1.9 Hz), 3.83 (3H, s), 3.61 (2H, s), 2.35 (3H, s).

STEP 2: A mixture of N-(4-bromo-3-methoxyphenyl)-3-oxobutanamide (1.21 g, 4.05 mmol) and polyphosphoric acid (10 g) was heated at 90° C. for 2 hours. The reaction mixture was allowed to cool to approximately 60° C. and then ice was added until a freely stirring mixture was achieved. The precipitate was isolated by filtration and dried under vacuum to yield 6-bromo-7-methoxy-4-methylquinolin-2(1H)-one (1.05 g, 3.90 mmol 96%) as a white solid. ¹H NMR (400 MHz, DMSO-d₆): δ ppm 7.87 (1H, s), 6.94 (1H, s), 6.27 (1H, s), 3.89 (3H, s), 2.38 (3H, s).

STEP 3: 6-bromo-7-methoxy-1,4-dimethylquinolin-2(1H)-one was prepared analogously to STEP 3 of Synthesis Example 4. Yield 52% as a pale yellow solid.

¹H NMR (400 MHz, DMSO-d₆): δ ppm 7.92 (1H, s), 7.02 (1H, s), 6.41 (1H, s), 4.02 (3H, s), 3.62 (3H, s), 2.39 (3H, d, J=0.5 Hz).

STEP 4: 6-amino-7-methoxy-1,4-dimethylquinolin-2(1H)-one was prepared analogously to STEP 4 of Synthesis Example 4. Yield 37% as an orange solid. ¹H NMR (400 MHz, DMSO-d₆): δ ppm 6.96 (1H, s), 6.87 (1H, s), 6.29 (1H, s), 4.82-4.76 (2H, br. s), 3.95 (3H, s), 3.59 (3H, s), 2.31 (3H, s).

Conversion of 6-amino-7-methoxy-1,4-dimethylquinolin-2(1H)-one to arylsulfonamide derivatives were carried out as described in General Procedure 1.

### 4-cyano-N-(7-methoxy-1,4-dimethyl-2-oxo-1,2-dihydroquinolin-6-yl)benzenesulfonamide (Compound 33)

¹H NMR (500 MHz, DMSO-d₆): δ ppm 10.05 (1H, br. s), 8.04 (2H, d, J=8.2 Hz), 7.83 (2H, d, J=8.5 Hz), 7.52 (1H, s), 6.84 (1H, s), 6.40 (1H, s), 3.56 (6H, m), 2.34 (3H, s).

### Synthesis Example 6

### Preparation of 6-amino-1,4-dimethylquinolin-2(1H)-one

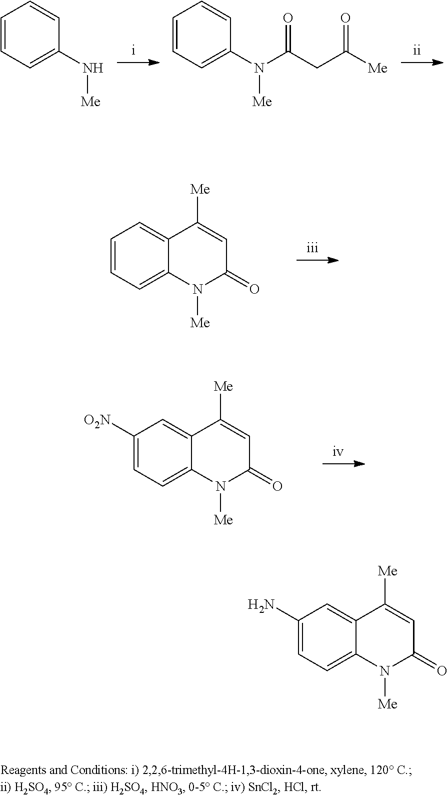 [SMILES: CNc1ccccc1 ; CC(=O)CC(=O)N(C)c1ccccc1 ; Cc1cc(=O)n(C)c2ccccc12 ; Cc1cc(=O)n(C)c2ccc([N+](=O)[O-])cc12 ; Cc1cc(=O)n(C)c2ccc(N)cc12]

STEP 1 Prepared according to the method of Qi et al.⁵

STEP 2 Prepared according to the method of Maiti et al.⁶

STEPS 3-4 Prepared according to the method of Kaslow et al.⁷

Conversion of 6-amino-1,4-dimethylquinolin-2(1H)-one to arylsulfonamide derivatives were carried out as described in General Procedure 1.

### 4-cyano-N-(1,4-dimethyl-2-oxo-1,2-dihydroquinolin-6-yl)benzenesulfonamide (Compound 30)

¹H NMR (400 MHz, DMSO-d6): δ ppm 8.04 (2H, d, J=8.3 Hz), 7.90 (2H, d, J=8.3 Hz), 7.48-7.39 (2H, m), 7.31 (1H, dd, J=9.0, 2.4 Hz), 6.54 (1H, s), 3.53 (3H, s), 2.31 (3H, s).

### 2-cyano-N-(1,4-dimethyl-2-oxo-1,2-dihydroquinolin-6-yl)benzenesulfonamide (Compound 46)

¹H NMR (400 MHz, DMSO-d₆) δ ppm 10.65 (1H, s), 8.91 (1H, d, J=5.3 Hz), 8.02-7.96 (3H, m), 7.50-7.24 (3H, m), 6.54 (1H, s), 3.52 (3H, s), 2.31 (3H, s).

### 3-cyano-N-(1,4-dimethyl-2-oxo-1,2-dihydroquinolin-6-yl)benzenesulfonamide (Compound 47)

¹H NMR (400 MHz, DMSO-d₆) δ ppm 10.53 (1H, s), 8.20 (1H, s), 8.11 (1H, d, J=7.8 Hz), 8.01 (1H, d, J=8.1 Hz), 7.77 (1H, t, J=7.6 Hz), 7.46-7.40 (2H, m), 7.32 (1H, dd, J=9.0, 2.4 Hz), 6.53 (1H, s), 3.52 (3H, s), 2.31 (3H, s).

### N-(1,4-dimethyl-2-oxo-1,2-dihydroquinolin-6-yl)-2,5-dimethylthiophene-3-sulfonamide (Compound 57)

¹H NMR (400 MHz, DMSO-d₆) δ ppm 7.46 (1H, d, J=9.1 Hz), 7.40 (1H, d, J=1.8 Hz), 7.33 (1H, dd, J=9.0, 1.9 Hz), 6.90 (1H, s), 6.54 (1H, s), 3.54 (3H, s), 2.41 (3H, s), 2.36-2.28 (6H, m).

### Synthesis Example 7

### Preparation of 6-amino-7-methoxy-1,3-dimethylquinolin-2(1H)-one

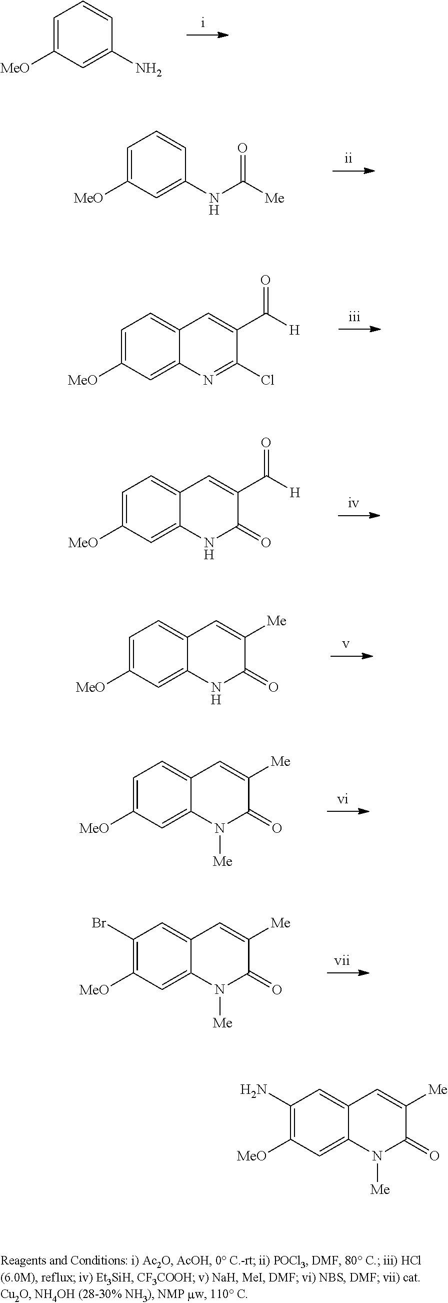

STEP 1: Prepared according to the method described in US2005/38076.⁸

STEP 2: Prepared according to the method of Cohn et al.⁹

STEPS 3-4: Prepared according to the method described in WO2006/112464.¹⁹

STEP 5: 7-methoxy-1,3-dimethylquinolin-2(1H)-one was prepared analogously to STEP 3 of Synthesis Example 4. Yield: 83%. ¹H NMR (400 MHz, CDCl₃): δ ppm 7.40 (1H, s), 7.33 (1H, d, J=8.6 Hz), 6.77-6.67 (2H, m), 3.84 (3H, s), 3.64 (3H, s).

STEP 6: 7-methoxy-1,3-dimethylquinolin-2(1H)-one (812 mg, 4 mmol 1 eq.) was dissolved in DMF (4 mL) and N-bromosuccinimide (855 mg, 4.8 mmol, 1.2 eq.) was added in one portion. The solution was stirred overnight at RT. Cold water (30 mL) was added and the resulting precipitate was filtered off to give 6-bromo-7-methoxy-1,3-dimethylquinolin-2(1H)-one (849 mg, 3.01 mmol, 75%) as an off white solid. ¹H NMR (500 MHz, CDCl₃): δ ppm 7.68 (1H, s), 7.40 (1H, s), 6.75 (1H, s), 4.03 (3H, s), 3.75 (3H, s), 2.25 (3H, s).

STEP 7: 6-amino-7-methoxy-1,3-dimethylquinolin-2(1H)-one was prepared analogously to STEP 4 of Synthesis Example 4. Yield: 61% of orange crystals. ¹H NMR (500 MHz, CDCl₃): δ ppm 7.40 (1H, s) 6.81 (1H, s) 6.71 (1H, s) 4.00 (3H, s) 3.75 (5H, m) 2.24 (3H, s).

Conversion of 6-amino-7-methoxy-1,3-dimethylquinolin-2(1H)-one to arylsulfonamide derivatives were carried out as described in General Procedure 1.

### N-(7-methoxy-1,3-dimethyl-2-oxo-1,2-dihydroquinolin-6-yl)benzenesulfonamide (Compound 21)

¹H NMR (500 MHz, DMSO-d₆): δ ppm 9.60 (1H, br. s), 7.72-7.66 (3H, m), 7.63-7.59 (1H, m), 7.55-7.47 (3H, m), 6.81 (1H, s), 3.60 (3H, s), 3.57 (3H, s), 2.08 (3H, s).

### 4-cyano-N-(7-methoxy-1,3-dimethyl-2-oxo-1,2-dihydroquinolin-6-yl)benzenesulfonamide (Compound 22)

¹H NMR (500 MHz, DMSO-d₆): δ ppm 9.98 (1H, br. s), 8.03 (2H, d, J=8.5 Hz), 7.82 (2H, d, J=8.2 Hz), 7.74 (1H, s), 7.51 (1H, s), 6.81 (1H, s), 3.61 (3H, s), 3.53 (3H, s), 2.09 (3H, s).

### 4-cyano-N-(7-methoxy-1,3-dimethyl-2-oxo-1,2-dihydroquinolin-6-yl)-2-methylbenzenesulfonamide (Compound 23)

¹H NMR (500 MHz, DMSO-d₆) δ ppm 9.97 (1H, s), 7.95 (1H, s), 7.74-7.66 (3H, m), 7.50 (1H, s), 6.78 (1H, s), 3.59 (3H, s), 3.52 (3H, s), 2.70 (3H, s), 2.08 (3H, s).

### N-(7-methoxy-1,3-dimethyl-2-oxo-1,2-dihydroquinolin-6-yl)-4-nitrobenzenesulfonamide (Compound 24)

¹H NMR (500 MHz, DMSO-d₆) δ ppm 10.06 (1H, s), 8.37 (2H, d, J=8.8 Hz), 7.91 (2H, d, J=8.8 Hz), 7.74 (1H, s), 7.51 (1H, s), 6.82 (1H, s), 3.61 (3H, s), 3.52 (3H, s), 2.09 (3H, s).

### 4-amino-N-(7-methoxy-1,3-dimethyl-2-oxo-1,2-dihydroquinolin-6-yl)benzenesulfonamide (Compound 25)

¹H NMR (500 MHz, DMSO-d₆) δ ppm 8.98 (1H, s), 7.69 (1H, s), 7.33 (2H, d, J=8.5 Hz), 6.84 (1H, s), 6.50 (2H, d, J=8.5 Hz), 5.92 (1H, s), 3.72 (3H, s), 3.63 (3H, s), 2.08 (3H, s).

### 4-cyano-2-ethoxy-N-(7-methoxy-1,3-dimethyl-2-oxo-1,2-dihydroquinolin-6-yl)benzenesulfonamide (Compound 26)

¹H NMR (500 MHz, DMSO-d₆) δ ppm 9.06 (1H, s), 7.75 (1H, d, J=3.8 Hz), 7.76-7.70 (2H, m), 7.50 (1H, s), 7.43 (1H, dd, J=8.0, 1.1 Hz), 6.84 (1H, s), 4.26 (2H, q, J=6.9 Hz), 3.67 (3H, s), 3.61 (3H, s), 2.07 (3H, s), 1.35 (3H, t, J=6.9 Hz).

### 4-cyano-2-methoxy-N-(7-methoxy-1,3-dimethyl-2-oxo-1,2-dihydroquinolin-6-yl)benzenesulfonamide (Compound 27)

¹H NMR (400 MHz, DMSO-d₆) δ ppm 9.35 (1H, br. s), 7.76 (1H, s), 7.70 (2H, d, J=7.8 Hz), 7.46 (1H, s), 7.43 (1H, dd, J=8.0, 1.4 Hz), 6.81 (1H, s), 3.97 (3H, s), 3.65 (3H, s), 3.60 (3H, s), 2.07 (3H, d, J=0.8 Hz).

### 2-methoxy-N-(7-methoxy-1,3-dimethyl-2-oxo-1,2-dihydroquinolin-6-yl)benzenesulfonamide (Compound 28)

¹H NMR (400 MHz, DMSO-d₆) δ ppm 8.82 (1H, s), 7.67 (1H, s), 7.61-7.53 (2H, m), 7.48 (1H, s), 7.21 (1H, d, J=7.8 Hz), 6.95 (1H, td, J=7.6, 1.0 Hz), 6.83 (1H, s), 3.92 (3H, s), 3.73 (3H, s), 3.59 (3H, s), 2.06 (3H, d, J=1.0 Hz).

### N-(7-methoxy-1,3-dimethyl-2-oxo-1,2-dihydroquinolin-6-yl)-4-methylbenzenesulfonamide (Compound 29)

¹H NMR (500 MHz, DMSO-d₆) δ ppm 9.51 (1H, s) 7.71 (1H, s) 7.58 (2H, d, J=8.2 Hz) 7.48 (1H, s) 7.32 (2H, d, J=8.2 Hz) 6.82 (1H, s) 3.65-3.56 (1H, m) 2.35 (3H, s) 2.08 (3H, s).

### N-(7-methoxy-1,3-dimethyl-2-oxo-1,2-dihydroquinolin-6-yl)-2-(trifluoromethoxy)benzenesulfonamide (Compound 67)

¹H NMR (500 MHz, DMSO-d₆) δ ppm 9.69 (1H, br. s), 7.75 (2H, d, J=7.9 Hz), 7.73-7.70 (1H, m), 7.56 (1H, d, J=7.6 Hz), 7.49 (1H, s), 7.44 (1H, t, J=7.6 Hz), 6.81 (1H, s), 3.61 (3H, s), 3.56 (3H, s), 2.07 (3H, s).

### Synthesis Example 8

### Preparation of 6-amino-5-methoxy-1-methylquinolin-2(1H)-one

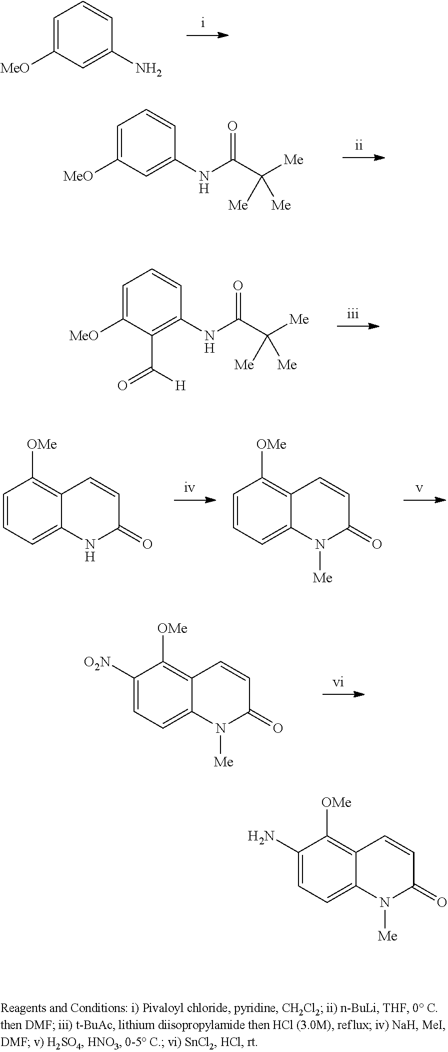

STEP 1: Prepared according to the method disclosed in WO2004/103996.¹¹

STEP 2: To a solution of diisopropylamine (904 mg, 1.25 mL, 8.94 mmol, 2.1 eq.) in dry Ether (12 mL) under an argon atmosphere at −78° C. was added n-butyllithium solution (1.6 M in hexanes, 5.6 mL, 2.1 eq.) and the solution was stirred for 30 minutes. Tert-butyl acetate (1036 mg, 1.20 mL, 8.94 mmol, 2.1 eq.) was added dropwise and the solution was allowed to stir for 30 minutes. N-(2-formyl-3-methoxyphenyl)pivalamide (1.0 g, 4.26 mmol, 1.0 eq.) in dry Ether (5 mL) was added dropwise and the bright yellow solution was allowed to warm to RT over 2 hours. Ammonium chloride solution (1.0 M, 20 mL) was added and the reaction mixture stirred for a further 10 minutes. The aqueous layer was separated and extracted twice with ether. The combined organic layers were washed with water and brine, dried over anhydrous MgSO₄, filtered and the solvent removed in vacuo.

STEP 3: To the crude residue from STEP 3 was added 1,4 dioxane (5 mL) and aqueous hydrochloric acid (3.0 M, 5 mL). The solution was heated at reflux for 4 hours. After cooling to room temperature the precipitated product was collected by filtration and dried under vacuum to yield 5-methoxyquinolin-2(1H)-one (605 mg, 3.46 mmol, 81% over 2 steps) as a fluffy white solid. ¹H NMR (400 MHz, DMSO-d₆): δ ppm 11.71 (1H, br. s) 8.03 (1H, d, J=9.6 Hz) 7.43 (1H, t, J=8.2 Hz) 6.90 (1H, d, J=8.3 Hz) 6.74 (1H, d, J=8.3 Hz) 6.42 (1H, d, J=9.9 Hz) 3.90 (3H, s).

STEP 4: 5-methoxy-1-methylquinolin-2(1H)-one was prepared analogously to STEP 3 of Synthesis Example 4. Yield: (325 mg, 1.72 mmol, 61%). ¹H NMR (400 MHz, DMSO-d₆): δ ppm 8.07 (1H, d, J=9.6 Hz) 7.57 (1H, t, J=8.5 Hz) 7.11 (1H, d, J=8.6 Hz) 6.87 (1H, d, J=8.1 Hz) 6.55 (1H, d, J=9.9 Hz) 3.93 (3H, s) 3.60 (3H, s).

STEP 5: To a solution of 5-methoxy-1-methylquinolin-2(1H)-one (270 mg, 1.43 mmol, 1 eq.) in concentrated sulfuric acid (5 mL) cooled to −5° C. was added potassium nitrate (144 mg, 1.43 mmol, 1 eq.) portionwise and the resulting yellow solution was stirred at this temperature for 1 hour before being allowed to warm to room temperature. The solution was poured over crushed ice, stirred for 10 minutes and the precipitate collected by filtration and dried under vacuum. The crude solid was purified by column chromatography (1:9-2:8, acetone: hexane) to give 5-methoxy-1-methyl-6-nitroquinolin-2(1H)-one as a pale yellow solid (48 mg, 0.2 mmol, 14%). ¹H NMR (400 MHz, DMSO-d₆): δ ppm 8.23 (1H, d, J=9.6 Hz) 8.15 (1H, d, J=9.9 Hz) 7.49 (1H, d, J=9.3 Hz) 6.79 (1H, d, J=9.9 Hz) 3.99 (3H, s) 3.66 (3H, s).

STEP 6: 6-amino-5-methoxy-1-methylquinolin-2(1H)-one was prepared analogously to STEP 4 of Synthesis Example 1 and used without further purification.

Conversion of 6-amino-5-methoxy-1-methylquinolin-2(1H)-one to arylsulfonamide derivatives were carried out as described in General Procedure 1.

### 4-cyano-N-(5-methoxy-1-methyl-2-oxo-1,2-dihydroquinolin-6-yl)benzenesulfonamide (Compound 16)

¹H NMR (400 MHz, DMSO-d₆): δ ppm 10.19 (1H, s) 8.10-8.02 (2H, m) 7.97-7.85 (3H, m) 7.39 (1H, d, J=9.1 Hz) 7.26 (1H, d, J=9.3 Hz) 6.61 (1H, d, J=9.6 Hz) 3.59-3.56 (6H, m).

### Synthesis Example 9

### Preparation of 6-amino-1-ethylquinolin-2(1H)-one

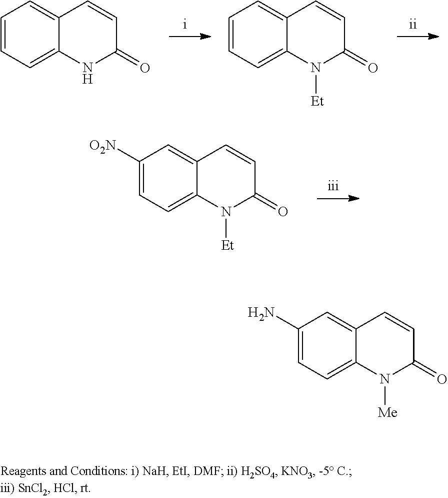 [SMILES: O=c1ccc2ccccc2[nH]1 ; CCn1c(=O)ccc2ccccc21 ; CCn1c(=O)ccc2cc([N+](=O)[O-])ccc21 ; Cn1c(=O)ccc2cc(N)ccc21]

STEP 1: To a solution of quinolin-2(1H)-one (1.0 g, 6.8 mmol, 1.0 eq.) in dry DMF (10 mL) under an argon atmosphere was added NaH (60%, 336 mg, 8.2 mmol, 1.2 eq.). Upon the completion of gas evolution, iodoethane (1.36 g, 700 μL, 8.70 mmol, 1.3 eq.) was added in 1 portion and the resulting solution was stirred overnight. Excess sodium hydride was quenched by the addition of water (4 mL) and the solvents were removed in vacuo. The residue was dissolved in ethyl acetate (40 mL), washed with water and then brine. The organic phase was dried over anhydrous MgSO₄, filtered and then concentrated in vacuo. Purification by column chromatography (1:1 ethyl acetate:hexanes) gave 1-ethylquinolin-2(1H)-one (759 mg, 4.39 mmol, 66%) as a colourless oil. ¹H NMR (400 MHz, DMSO-d₆): δ ppm 7.90 (1H, d, J=9.6 Hz), 7.73 (1H, d, J=7.6 Hz), 7.66-7.56 (2H, m), 7.27 (1H, t, J=7.8 Hz), 6.61 (1H, d, J=9.6 Hz), 4.29 (2H, q, J=7.1 Hz), 1.22 (3H, t, J=7.1 Hz).

STEP 2: To a suspension of 1-ethylquinolin-2(1H)-one (759 mg, 4.39 mmol, 1.0 eq.) in concentrated H₂SO₄ (5 mL) at −5° C. was added KNO₃ (444 mg, 4.39 mmol, 1.0 eq.)

portionwise and the resulting yellow solution was stirred at this temperature for 1 hour before being allowed to warm to room temperature. The solution was poured over crushed ice, stirred for 10 minutes and the precipitate collected by filtration and dried under vacuum to give 1-ethyl-6-nitroquinolin-2(1H)-one (804 mg, 3.68 mmol, 84%) as a pale yellow solid. ¹H NMR (400 MHz, DMSO-d₆): δ ppm 8.71 (2H, d, J=2.8 Hz), 8.37 (2H, dd, J=9.4, 2.5 Hz), 8.13 (2H, d, J=9.6 Hz), 7.77 (2H, d, J=9.6 Hz), 6.77 (2H, d, J=9.6 Hz), 4.30 (4H, q, J=7.1 Hz), 1.22 (6H, t, J=7.1 Hz).

STEP 3: To a suspension of 1-ethyl-6-nitroquinolin-2(1H)-one (500 mg, 2.29 mmol, 1.0 eq.) in concentrated HCl (15 mL) was added SnCl₂ (2.17 g, 11.46 mmol, 5.0 eq.) and the resulting suspension was stirred overnight. Aqueous sodium hydroxide (2.0 M) was added until all solids had dissolved and the solution was bright yellow (˜pH 10). The aqueous solution was extracted with DCM (3×250 mL). The organic layers were combined and the solvent removed in vacuo to give 6-amino-1-ethylquinolin-2(1H)-one (428 mg, 2.29 mmol, 100%) as a bright yellow solid. ¹H NMR (400 MHz, DMSO-d₆): δ ppm 7.67 (1H, s), 7.30 (1H, d, J=9.1 Hz), 6.96 (1H, dd, J=9.1, 2.5 Hz), 6.80 (1H, d, J=2.5 Hz), 6.47 (1H, d, J=9.4 Hz), 5.08 (2H, br. s), 4.20 (2H, q, J=7.1 Hz), 1.18 (3H, t, J=7.1 Hz).

Conversion of 6-amino-1-ethylquinolin-2(1H)-one to arylsulfonamide derivatives were carried out as described in General Procedure 1.

### 4-cyano-N-(1-ethyl-2-oxo-1,2-dihydroquinolin-6-yl)benzenesulfonamide (Compound 35)

¹H NMR (400 MHz, DMSO-d₆): δ ppm 10.58 (1H, br. s), 8.04 (2H, d, J=8.3 Hz), 7.90 (2H, d, J=8.3 Hz), 7.85 (1H, d, J=9.6 Hz), 7.50 (1H, d, J=9.1 Hz), 7.44 (1H, d, J=2.3 Hz), 7.29 (1H, dd, J=9.0, 2.4 Hz), 6.58 (1H, d, J=9.6 Hz), 4.20 (2H, q, J=6.9 Hz), 1.16 (3H, t, J=6.9 Hz).

### 4-cyano-N-(1-ethyl-3-methyl-2-oxo-1,2-dihydroquinolin-6-yl)benzenesulfonamide (Compound 58)

¹H NMR (500 MHz, DMSO-d₆) δ ppm 10.55 (1H, s), 8.04 (2H, d, J=8.2 Hz), 7.89 (2H, d, J=8.5 Hz), 7.73 (1H, s), 7.46 (1H, d, J=9.1 Hz), 7.35 (1H, d, J=2.2 Hz), 7.23 (1H, dd, J=9.1, 2.5 Hz), 4.22 (2H, q, J=6.9 Hz), 2.09 (3H, s), 1.16 (3H, t, J=6.9 Hz).

### Synthesis Example 10

### Preparation of N-(4-cyanophenyl)-1-methyl-2-oxo-1,2-dihydroquinoline-6-sulfonamide (Compound 38)

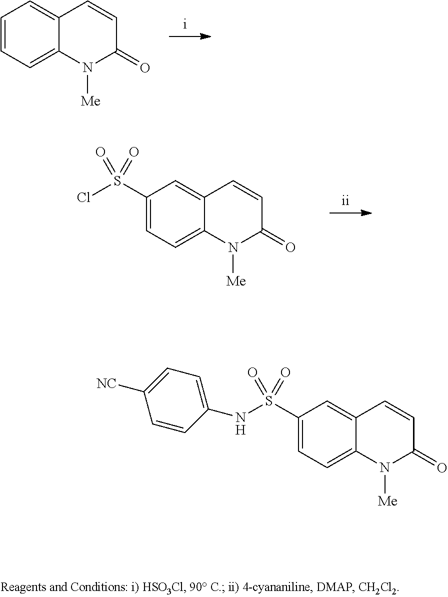 [SMILES: Cn1c(=O)ccc2ccccc21 ; Cn1c(=O)ccc2cc(S(=O)(=O)Cl)ccc21 ; Cn1c(=O)ccc2cc(S(=O)(=O)Nc3ccc(C#N)cc3)ccc21]

STEP 1: A mixture of 1-methylquinolin-2(1H)-one (1.0 g, 6.29 mmol, 1.0 eq.) in chlorosulfonic acid (5 mL) was heated at 90° C. for 2 hours. After cooling to RT the solution was poured over crushed ice and the resulting precipitate filtered and dried under vacuum to give 1-methyl-2-oxo-1,2-dihydroquinoline-6-sulfonyl chloride (1.4 g, 5.22 mmol, 83%) as a pale brown solid. ¹H NMR (400 MHz, CDCl₃): δ ppm 8.28 (1H, d, J=2.0 Hz), 8.19 (1H, dd, J=9.1, 2.3 Hz), 7.79 (1H, d, J=9.6 Hz), 7.56 (1H, d, J=9.1 Hz), 6.89 (1H, d, J=9.6 Hz), 3.80 (3H, s).

STEP 2: To a solution of 4-cyanoaniline (100 mg, 0.84 mmol, 1.0 eq.) in DCM (5 mL) was added 1-methyl-2-oxo-1,2-dihydroquinoline-6-sulfonyl chloride (326 mg, 1.26 mmol, 1.5 eq.) and 4-(dimethylamino)pyridine (10 mg, 0.084 mmol, 0.1 eq). The mixture was stirred for 3 days after which time the solvent was removed in vacuo and the residue purified by column chromatography (9:1-10:0, ethyl acetate:hexane) to give N-(4-cyanophenyl)-1-methyl-2-oxo-1,2-dihydroquinoline-6-sulfonamide (114 mg, 0.33 mmol, 40%) as a white solid.

### N-(4-cyanophenyl)-1-methyl-2-oxo-1,2-dihydroquinoline-6-sulfonamide (Compound 38)

¹H NMR (400 MHz, DMSO-d₆): δ ppm 11.11 (1H, br. s), 8.31 (1H, d, J=2.0 Hz), 8.08 (1H, d, J=9.6 Hz), 7.96 (1H, dd, J=9.0, 2.2 Hz), 7.70 (3H, d, J=8.8 Hz), 7.27 (2H, d, J=8.6 Hz), 6.74 (1H, d, J=9.4 Hz), 3.61 (3H, s).

The following compounds of the invention were synthesised using methods analogous to those set out in Synthesis Example 10 with corresponding reagents in step 2.

### N-(2-cyanophenyl)-1-methyl-2-oxo-1,2-dihydroquinoline-6-sulfonamide (Compound 60)

¹H NMR (400 MHz, DMSO-d₆) δ ppm 10.61 (1H, br. s), 8.14 (1H, d, J=2.0 Hz), 8.05 (1H, d, J=9.6 Hz), 7.90 (1H, dd, J=9.1, 2.3 Hz), 7.82 (1H, dd, J=7.8, 1.3 Hz), 7.73 (1H, d, J=9.1 Hz), 7.59 (1H, td, J=7.8, 1.5 Hz), 7.39 (1H, t, J=8.0 Hz), 7.07 (1H, d, J=8.1 Hz), 6.74 (1H, d, J=9.4 Hz), 3.64 (3H, s).

### N-(3-cyanophenyl)-1-methyl-2-oxo-1,2-dihydroquinoline-6-sulfonamide (Compound 61)

¹H NMR (400 MHz, DMSO-d₆) δ ppm 8.63 (1H, d, J=2.0 Hz), 8.47 (1H, dd, J=9.3, 2.8 Hz), 8.34 (1H, dd, J=8.1, 2.0 Hz), 8.28 (1H, d, J=2.5 Hz), 7.86 (1H, d, J=8.1 Hz), 7.82 (1H, d, J=9.3 Hz), 6.73 (1H, s), 3.66 (3H, s).

### Synthesis Example 11

### Preparation of 4-cyano-N-methyl-N-(1-methyl-2-oxo-1,2-dihydroquinolin-6-yl)benzenesulfonamide (Compound 34)

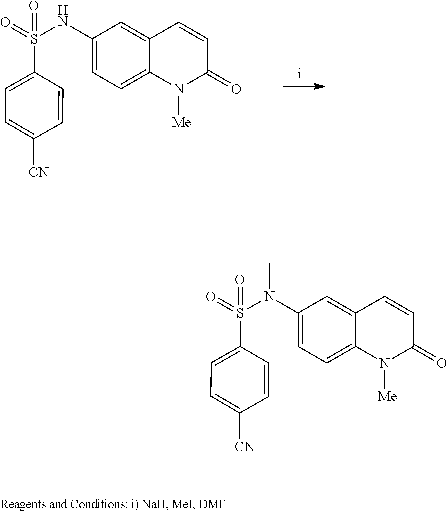 [SMILES: Cn1c(=O)ccc2cc(NS(=O)(=O)c3ccc(C#N)cc3)ccc21 ; CN(c1ccc2c(ccc(=O)n2C)c1)S(=O)(=O)c1ccc(C#N)cc1]

To a solution of 4-cyano-N-(1-methyl-2-oxo-1,2-dihydroquinolin-6-yl)benzenesulfonamide (50 mg, 0.15 mmol, 1 eq.) in dry DMF (2 mL) under an argon atmosphere was added sodium hydride (60%, 7 mg, 0.18 mmol, 1.2 eq.). Upon the completion of gas evolution, iodomethane (11 μL, 0.18 mmol, 1.2 eq.) was added in 1 portion and the resulting solution was stirred overnight. Excess sodium hydride was quenched by the addition of water (1 mL) and the solvents were removed in vacuo. The residue was dissolved in ethyl acetate (15 mL), washed with water and then brine. The organic phase was dried over anhydrous MgSO₄, filtered and then concentrated in vacuo. to give 4-cyano-N-methyl-N-(1-methyl-2-oxo-1,2-dihydroquinolin-6-yl)benzenesulfonamide (46 mg, 0.13 mmol, 88%) as a white solid.

### 4-cyano-N-methyl-N-(1-methyl-2-oxo-1,2-dihydroquinolin-6-yl)benzenesulfonamide (Compound 34)

¹H NMR (400 MHz, DMSO-d6) δ ppm 8.08 (2H, d, J=8.3 Hz) 7.85 (1H, d, J=9.6 Hz) 7.70 (2H, d, J=8.6 Hz) 7.56-7.46 (2H, m) 7.37 (1H, dd, J=9.1, 2.5 Hz) 6.65 (1H, d, J=9.6 Hz) 3.61 (3H, s) 3.22 (3H, s)

The following compound of the invention was synthesised using methods analogous to Synthesis Example 11 with corresponding starting material.

### 4-cyano-N-(1,3-dimethyl-2-oxo-1,2-dihydroquinolin-6-yl)-2-methoxy-N-methylbenzenesulfonamide (Compound 59)

¹H NMR (500 MHz, DMSO-d₆) δ ppm 6.99 (1H, d, J=8.2 Hz) 6.89 (1H, s), 6.78 (1H, s), 6.71-6.60 (3H, m), 6.53 (1H, d, J=8.2 Hz), 3.07 (3H, s) 2.91 (3H, s), 2.61 (3H, s).

### 4-cyano-N-(7-methoxy-1-methyl-2-oxo-1,2-dihydroquinolin-6-yl)-N-methylbenzenesulfonamide (Compound 68)

¹H NMR (400 MHz, DMSO-d₆) δ ppm 8.09 (1H, d, J=8.6 Hz), 7.86 (1H, d, J=9.6 Hz), 7.82 (2H, d, J=8.6 Hz), 7.66 (1H, s), 6.89 (1H, s), 6.49 (1H, d, J=9.3 Hz), 3.62 (3H, s), 3.50 (3H, s), 3.19 (3H, s).

### Synthesis Example 12

### Preparation of 4-cyano-N-(7-methoxy-1,3,4-trimethyl-2-oxo-1,2-dihydroquinolin-6-yl)benzenesulfonamide (Compound 50)

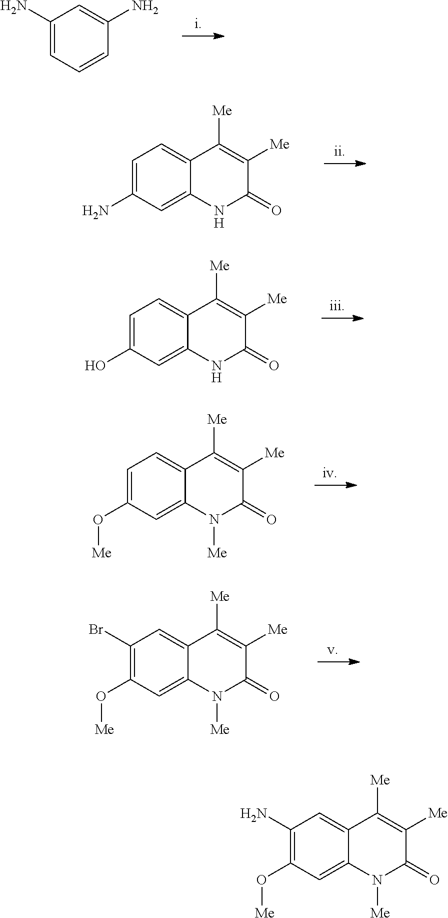

STEP 1 Prepared according to the method of Yamada et al.²²

STEP 2 Prepared according to the method of Chilin et al.²³

STEP 3 Prepared by methylation using standard conditions (DMF, NaH and MeI).

STEPS 4 and 5 C6-bromination then C6-aminolysis was carried out according to Synthesis Example 7 (steps 6 and 7).

Conversion of 6-amino-7-methoxy-1,3-dimethylquinolin-2(1H)-one to Compound 50 was carried out as described in General Procedure 1.

### 4-cyano-N-(7-methoxy-1,3,4-trimethyl-2-oxo-1,2-dihydroquinolin-6-yl)benzenesulfonamide (Compound 50)

¹H NMR (500 MHz, CDCl₃) δ ppm 9.12 (1H, br. s.), 7.68 (2H, d, J=7.8 Hz), 7.63 (2H, d, J=7.8 Hz), 7.56 (1H, s), 7.38 (1H, s), 3.60 (3H, s), 3.54 (3H, d, J=1.3 Hz), 2.24 (3H, d, J=1.4 Hz) 2.13 (3H, s).

The following compounds of the invention were synthesised using methods analogous to those described in Wu et al.²⁴

### 4-cyano-N-(3-ethyl-1-methyl-2-oxo-1,2-dihydroquinolin-6-yl)benzenesulfonamide (Compound 44)

¹H NMR (400 MHz, DMSO-d₆) δ ppm 10.57 (1H, s), 8.04 (2H, d, J=8.6 Hz), 7.88 (1H, d, J=8.6 Hz), 7.69 (1H, s), 7.46-7.35 (2H, m), 7.22 (1H, dd, J=8.8, 2.5 Hz), 3.57 (3H, s), 1.15 (3H, t, J=7.5 Hz) (Note: CH₂ overlaps with DMSO).

### 4-cyano-N-(3-ethyl-1-methyl-2-oxo-1,2-dihydroquinolin-6-yl)-2-methoxybenzenesulfonamide (Compound 45)

¹H NMR (400 MHz, DMSO-d₆) δ ppm 10.37 (1H, s), 7.92 (1H, d, J=8.1 Hz), 7.80 (1H, d, J=1.3 Hz), 7.71 (1H, s), 7.54 (1H, dd, J=8.1, 1.5 Hz), 7.47-7.40 (2H, m), 7.31 (1H, dd, J=9.0, 2.4 Hz), 4.04 (3H, s), 3.61 (3H, s), 1.20 (3H, t, J=7.3 Hz) (Note: CH₂ overlaps with DMSO).

Other compounds of the invention were synthesised using methods analogous to those set out above.

### Biological Methods

Compounds were tested using the commercially available BROMOscan™ Platform (DiscoveRx): http://www.discoverx.com/technologies-platforms/competitive-binding-technology/bromoscan-technology-platform/bromoscan-assay-process.

### Protocol Description

Bromodomain assays. T7 phage strains displaying bromodomains were grown in parallel in 24-well blocks in an E. coli host derived from the BL21 strain. E. coli were grown to log-phase and infected with T7 phage from a frozen stock (multiplicity of infection=0.4) and incubated with shaking at 32° C. until lysis (90-150 minutes). The lysates were centrifuged (5,000×g) and filtered (0.2 μm) to remove cell debris. Streptavidin-coated magnetic beads were treated with biotinylated small molecule or acetylated peptide ligands for 30 minutes at room temperature to generate affinity resins for bromodomain assays. The liganded beads were blocked with excess biotin and washed with blocking buffer (SeaBlock (Pierce), 1% BSA, 0.05% Tween 20, 1 mM DTT) to remove unbound ligand and to reduce non-specific phage binding. Binding reactions were assembled by combining bromodomains, liganded affinity beads, and test compounds in 1× binding buffer (17% SeaBlock, 0.33×PBS, 0.04% Tween 20, 0.02% BSA, 0.004% Sodium azide, 7.4 mM DTT). Test compounds were prepared as 1000× stocks in 100% DMSO and subsequently diluted 1:10 in monoethylene glycol (MEG) to create stocks at 100× the screening concentration (resulting stock solution is 10% DMSO/90% MEG). The compounds were then diluted directly into the assays such that the final concentration of DMSO and MEG were 0.1% and 0.9%, respectively. All reactions were performed in polystyrene 96-well plates in a final volume of 0.135 ml. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 2 μM non-biotinylated affinity ligand) and incubated at room temperature with shaking for 30 minutes. The bromodomain concentration in the eluates was measured by qPCR.

### Compound Handling

An 11-point 3-fold serial dilution of each test compound was prepared in 100% DMSO at 1000× final test concentration. This serial is then diluted to 100× in ethylene glycol and subsequently diluted to 1× in the assay (final DMSO concentration=0.1%, Ethylene glycol concentration=0.9%). Most Kds were determined using a compound top concentration=10,000 nM. If the initial Kd determined was <0.169 nM (the lowest concentration tested), the measurement was repeated with a serial dilution starting at a lower top concentration. A Kd value reported as 40,000 nM indicates that the Kd was determined to be >10,000 nM.

### Binding Constants (Kds)

Binding constants (Kds) were calculated with a standard dose-response curve using the Hill equation:

$$Response = Background + \frac{Signal - Background}{\begin{matrix}1 + \\ ( {Kd}^{Hill Slope} / {Dose}^{Hill Slope} )\end{matrix}}$$

The Hill Slope was set to −1. Curves were fitted using a non-linear least square fit with the Levenberg-Marquardt algorithm.

BROMOscan™ uses the same assay technology as KINOMEscan™. For a more detailed description of this assay technology, see Fabian et al.12

### Biological Data

Against BRPF1B all compounds tested had K_{D} values below 2500 nM, with many having much lower K_{D} values, for example below 1000 nM, below 500 nM, below 250 nM, below 100 nM or below 50 nM. Particular compounds of the invention had K_{D} values below 20 nM or below 10 nM.

Compounds of the invention showed selectivity for BRPF1B over other bromodomain proteins. All compounds tested had K_{D} values for BRD1, for example, which were higher than those for BRPF1B, for example at least 5 times higher, at least 10 times higher, at least 20 times higher, or at least 30 times higher than for BRPF1B.

Similarly, where tested, compounds of the invention showed higher K_{D} values for BRPF3, BRD9 and BRD7 than for BRPF1B.

Data for certain representative compounds is set out in the table below.

| Compound | K_{D} (nM)^{a,b} | | | | |
|---|---|---|---|---|---|
| Ref | BRPF1B | BRD1 | BRPF3 | BRD9 | BRD7 |
| 62 | 5 | | | | |
| 19 | 7.9 (n = 4) | 48 | 260 | 310 | 82 |
| P-001 | 9 | 130 | | | |
| 49 | 10 | | | | |
| 27 | 12 | 80 | 190 | 170 | |
| 22 | 15 | 100 | 280 | | |
| 21 | 17 | 190 | 590 | | |
| 30 | 24 | 150 | | | |
| 10 | 29 | 380 | 2200 | | |
| 56 | 31 | | | | |
| 14 | 39 | | | | |
| 4 | 43 | 200 | 1000 | 910 | |
| 9 | 44 | 230 | | | |
| 16 | 46 | 210 | | | |
| 35 | 47 | 390 | | | |
| 67 | 48 | | | | |
| 59 | 52 | | | | |
| 11 | 53 | 520 | 2200 | 2700 | |
| 13 | 55 | | | | |
| 3 | 56 | 270 | | | |
| 33 | 60 (n = 4) | 1800 | 9300 | 890 | 21000 |
| 1 | 65 | 1400 | 7600 | | |
| 45 | 66 | | | | |
| 38 | 100 | 150 | | | |
| 50 | 140 | | | | |
| 18 | 210 | 840 | | | |
| 55 | 380 | | | | |
| 25 | 420 | | | | |
| 2 | 430 | 1700 | | | |
| 54 | 520 | | | | |
| 34 | 910 | 6700 | | | |
| 53 | 1100 | | | | |
| 52 | 1200 | | | | |
| 51 | 2200 | | | | |

^{a}Screened at DiscoveRx using their biochemical BROMOscan ™ platform (n = 2).
^{b}Selectivity data for bromodomains: BRD4(1), CREBBP, PCAF available for some examples (K_{d} minimum >2000 nM, typically >10,000 nM)

### REFERENCES

1. You, L. et al; “Expression atlas of the multivalent epigenetic regulator BRPF1 and its requirement for survival of mouse embryos”; Epigenetics., 2014, 9(6), 860.
2. H. Shima et al.; “BRPF1 is critical for leukemogenosis associated with MOZ-TIF2 fusion”; Int. J. Haematol., 2014, 99(1), 21.
3. Ullah et al.; “Molecular architecture of quartet MOZ/MORF Histone Acetyltransferase Complexes” Mol. Cell. Biol., 2008, 22, 6828.
4. Golpon, H. A., et al; “HOX Genes in Human Lung: Altered Expression in Primary Pulmonary Hypertension and Emphysema”; Am. J. Path., 2001, 158(3), 955.
5. Cillo C, et al; “Homeobox genes and cancer”; Exp Cell Res., 1999, 248, 1.
6. Tiberio C, et al; “HOX gene expression in human small-cell lung cancers xenografted into nude mice”; Int. J. Cancer, 1994, 58, 608.
7. You, L. et al; “The chromatin regulator Brpf1 regulates embryo development and cell proliferation”; J. Biol. Chem, 2015, jbc.M115.643189.
8. You, L. et al; “The Lysine Acetyltransferase Activator Brpf1 Governs Dentate Gyrus Development through Neural Stem Cells and Progenitors”; PLoS Genet., 2015, 11(3), e1005034.
9. You, L. et al; “Deficiency of the chromatin regulator Brpf1 causes abnormal brain development”; J. Biol. Chem, 2015, 290, 7114.
10. International patent publication WO2013/027168 (Pfizer Inc); published 28 Feb. 2013.
11. Fernandez et al Synthesis 2001 239-242.
12. Qi, H., Z. Yang, et al. (2011). “Synthesis of 3-Alkoxy/Aryloxy-β-lactams Using Diazoacetate Esters as Ketene Precursors Under Photoirradiation.” Synthesis 2011(05): 723-730.
13. Maiti, A., P. V. N. Reddy, et al. (2009). “Synthesis of Casimiroin and Optimization of Its Quinone Reductase 2 and Aromatase Inhibitory Activities.” Journal of Medicinal Chemistry 52(7): 1873-1884.
14. Kaslow, C. E. and D. J. Cook (1945). “N-Substituted 4-Methylcarbostyrils.” Journal of the American Chemical Society 67(11): 1969-1972.
15. US patent publication US2005/0038076 (Garst et al); published 17 Feb. 2005.
16. Cohn et al; Journal of the Chemical Society, Perkin Transactions 1, 1981, 520-1530
17. International patent publication WO2006/112464 (Otsuka Pharma Co Ltd); published 26 Oct. 2006.
18. International patent publication WO2004/103996 (Boehringer Ingelheim Int.); published 2 Dec. 2004.
19. Fabian, M. A. et al: “A small molecule-kinase interaction map for clinical kinase inhibitors”, Nat. Biotechnol., 23, 329-336 (2005).
20. van Oeveren, A. et al., 2006, “Discovery of 6-N,N-Bis(2,2,2-trifluoroethyl)amino-4-trifluoromethylquinolin-2(1H)-one as a Novel Selective Androgen Receptor Modulator”, Journal of Medicinal Chemistry, Vol. 49, No. 21, pp. 6143-6146.
21. Manimaran et al., 1979, Indian Journal of Organic Chemistry, Vol. 88, pp. 125-129.
22. Yamada et al., 2010, “Fluorescent retinoid X receptor ligands for fluorescence polarization assay”, Bioorganic & Medicinal Chemistry Letters, Vol. 20, No. 17, pp. 5143-5146.
23. Chilin et al., 1991, “New synthesis of pyrrolo [3,2,11]quinolin-4-one derivatives”, The Journal of Organic Chemistry, Vol. 56, No. 3, pp. 980-983.
24. Wu et al., 2014, “Design and chemoproteomic functional characterization of a chemical probe targeted to bromodomains of BET family proteins”, Med. Chem. Comm., Vol. 5, No. 12, pp. 1871-1878.

1. A compound of general formula I: 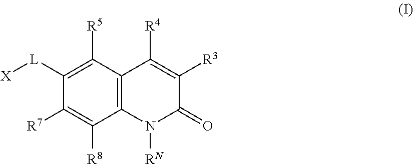
wherein:
R³ is selected from —R^{3A} and —OR^{3B} wherein R^{3A} and R^{3B} are each independently selected from hydrogen, C₁₋₄alkyl and C₁₋₄haloalkyl;
R⁴ is selected from —R^{4A} and —OR^{4B} wherein R^{4A} and R^{4B} are each independently selected from hydrogen, C₁₋₄alkyl and C₁₋₄haloalkyl;
R⁵ is selected from —R^{5A} and —OR^{5B} wherein R^{5A} is independently selected from hydrogen, halo, C₁₋₄alkyl, C₂₋₄alkenyl, C₂₋₄alkynyl, C₃₋₆cycloalkyl and C₁₋₄haloalkyl, and wherein R^{5B} is independently selected from hydrogen, C₁₋₄alkyl, C₃₋₆cycloalkyl and C₁₋₄haloalkyl;
R⁷ is selected from —R^{7A} and —OR^{7B} wherein R^{7A} and R^{7B} are each independently selected from hydrogen, C₁₋₄alkyl, C₃₋₆cycloalkyl, and C₁₋₄haloalkyl;
R⁸ is selected from —R^{8A} and —OR^{8B} wherein R^{8A} is independently selected from hydrogen, halo, C₁₋₄alkyl, and C₁₋₄haloalkyl, and wherein R^{8B} is independently selected from hydrogen, C₁₋₄alkyl and C₁₋₄haloalkyl;
R^{N} is selected from C₁₋₄alkyl, C₁₋₄haloalkyl, R^{Z}, and —Z^{N}—R^{Z} wherein Z^{N} is C₁₋₄alkylene and each R^{Z} is independently C₃₋₆cycloalkyl;
L is a sulfonamide linker;
X is selected from aryl, C₁₋₆alkyl and C₃₋₆cycloalkyl and is optionally substituted.

2. A compound according to claim 1 wherein L is a sulfonamide linker selected from: 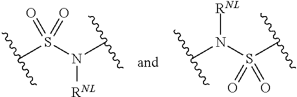
wherein R^{NL} is selected from hydrogen and C₁₋₄alkyl.

3. A compound according to claim 2 which is a compound of formula (IIa):

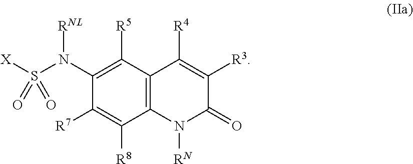

4. A compound according to claim 2 wherein R^{NL} is hydrogen or -Me.

5. A compound according to claim 1 wherein R^{N} is selected from -Me and -Et.

6. A compound according to claim 1 wherein X is aryl.

7. A compound according to claim 6 wherein X is selected from C₆₋₂₀carboaryl and C₅₋₁₂heteroaryl.

8. A compound according to claim 7 wherein X is selected from phenyl, naphthyl, furanyl, thienyl, pyrrolyl, imidazolyl, pyrazolyl, triazolyl, tetrazolyl, oxazolyl, isoxazolyl, thiazolyl, isothiazolyl, pyridyl, pyrimidinyl, pyridazinyl, indolyl, isoindolyl, benzofuranyl, isobenzofuranyl, benzothienyl, isobenzothienyl, indazolyl, benzimidazolyl, benzothiazolyl, benzoisothiazolyl, benzoxazolyl, benzoisoxazolyl, quinolinyl, isoquinolinyl, cinnolinyl, or quinazolinyl.

9. A compound according to claim 7 wherein X is phenyl.

10. A compound according to claim 9, wherein X is phenyl, substituted with at least one group R^{X} wherein each R^{X} is independently selected from halo, C₁₋₄alkyl, C₁₋₄haloalkyl, —OR^{XO}, —C(═O)OR^{XO}, —N(R^{XN})₂, —C(═O)N(R^{XN})₂, —SR^{XS}, —S(═O)R^{XS}, —S(═O)₂R^{XS}, —SO₂OR^{XO}, —SO₂N(R^{XN})₂, —CN, and —NO₂; wherein each R^{XO}, R^{XN} and R^{XS} is selected from hydrogen, C₁₋₄alkyl and C₁₋₄haloalkyl.

11. A compound according to claim 10, wherein each R^{X} is independently selected from —Cl, —CN, —OMe and -Me.

12. A compound according to claim 10, wherein R^{X} is independently —CN.

13. A compound according to claim 10, wherein X is 4-cyanophenyl.

14. A compound according to claim 1 wherein X is C₃₋₆cycloalkyl.

15. A compound according to claim 14 wherein X is cyclohexyl (c-Hex).

16. A compound according to claim 1 wherein R³ is independently selected from hydrogen, -Et, and -Me.

17. A compound according to claim 1 wherein R³ is independently selected from hydrogen and -Me.

18. A compound according to claim 1 wherein R⁴ is independently selected from hydrogen and -Me.

19. A compound according to claim 1, wherein R⁵ is independently selected from hydrogen, halo, and —OMe.

20. A compound according to claim 1, wherein R⁷ is independently selected from hydrogen and —OMe.

21. A compound according to claim 1, wherein R⁸ is independently selected from hydrogen and —F.

22. A compound according to claim 1 which is a compound selected from:

| Compound | | |
|---|---|---|
| Ref | Structure | IUPAC name |
| 2 | 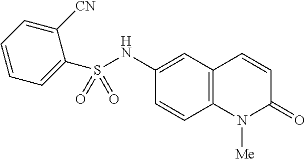 [SMILES: Cn1c(=O)ccc2cc(NS(=O)(=O)c3ccccc3C#N)ccc21] | 2-cyano-N-(1-methyl-2-oxo-6- quinolyl)benzenesulfonamide |
| 3 | 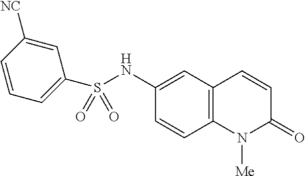 [SMILES: Cn1c(=O)ccc2cc(NS(=O)(=O)c3cccc(C#N)c3)ccc21] | 3-cyano-N-(1-methyl-2-oxo-6- quinolyl)benzenesulfonamide |
| 4 | 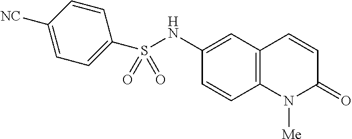 [SMILES: Cn1c(=O)ccc2cc(NS(=O)(=O)c3ccc(C#N)cc3)ccc21] | 4-cyano-N-(1-methyl-2-oxo-6- quinolyl)benzenesulfonamide |
| 9 | 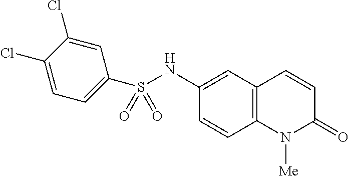 [SMILES: Cn1c(=O)ccc2cc(NS(=O)(=O)c3ccc(Cl)c(Cl)c3)ccc21] | 3,4-dichloro-N-(1-methyl-2-oxo-6- quinolyl)benzenesulfonamide |
| 10 | 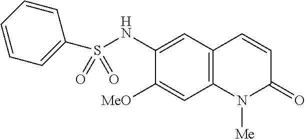 [SMILES: COc1cc2c(ccc(=O)n2C)cc1NS(=O)(=O)c1ccccc1] | N-(7-methoxy-1-methyl-2-oxo-6- quinolyl)benzenesulfonamide |
| 11 | 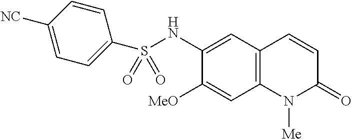 [SMILES: COc1cc2c(ccc(=O)n2C)cc1NS(=O)(=O)c1ccc(C#N)cc1] | 4-cyano-N-(7-methoxy-1-methyl-2-oxo- 6-quinolyl)benzenesulfonamide |
| 16 | 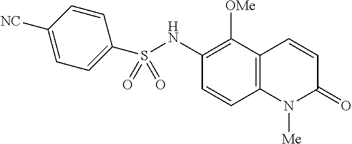 [SMILES: COc1c(NS(=O)(=O)c2ccc(C#N)cc2)ccc2c1ccc(=O)n2C] | 4-cyano-N-(5-methoxy-1-methyl-2-oxo- 6-quinolyl)benzenesulfonamide |
| 18 | 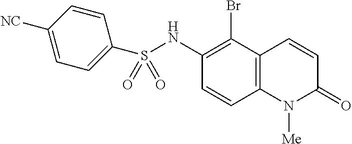 [SMILES: Cn1c(=O)ccc2c(Br)c(NS(=O)(=O)c3ccc(C#N)cc3)ccc21] | 4-cyano-N-(5-bromo-1-methyl-2-oxo-6- quinolyl)benzenesulfonamide |
| 19 | 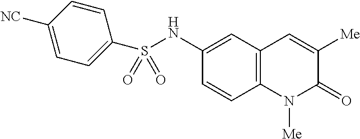 [SMILES: Cc1cc2cc(NS(=O)(=O)c3ccc(C#N)cc3)ccc2n(C)c1=O] | 4-cyano-N-(1,3-dimethyl-2-oxo-6- quinolyl)benzenesulfonamide |
| 21 | 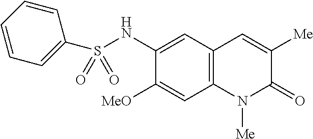 [SMILES: COc1cc2c(cc1NS(=O)(=O)c1ccccc1)cc(C)c(=O)n2C] | N-(1,3-dimethyl-7-methoxy-2-oxo-6- quinolyl)benzenesulfonamide |
| 22 | 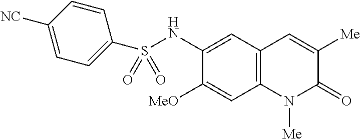 [SMILES: COc1cc2c(cc1NS(=O)(=O)c1ccc(C#N)cc1)cc(C)c(=O)n2C] | 4-cyano-N-(1,3-dimethyl-7-methoxy-2- oxo-6-quinolyl)benzenesulfonamide |
| 30 | 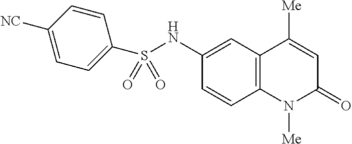 [SMILES: Cc1cc(=O)n(C)c2ccc(NS(=O)(=O)c3ccc(C#N)cc3)cc12] | 4-cyano-N-(1,4-dimethyl-2-oxo-6- quinolyl)benzenesulfonamide |
| 33 | 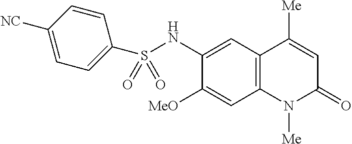 [SMILES: COc1cc2c(cc1NS(=O)(=O)c1ccc(C#N)cc1)c(C)cc(=O)n2C] | 4-cyano-N-(1,4-dimethyl-7-methoxy-2- oxo-6-quinolyl)benzenesulfonamide |
| 34 | 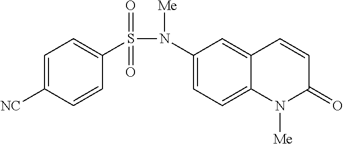 [SMILES: CN(c1ccc2c(ccc(=O)n2C)c1)S(=O)(=O)c1ccc(C#N)cc1] | 4-cyano-N-methyl-N-(1-methyl-2-oxo-6- quinolyl)benzenesulfonamide |
| 35 | 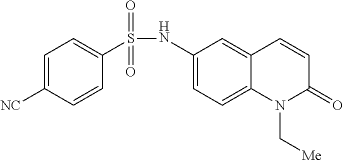 [SMILES: CCn1c(=O)ccc2cc(NS(=O)(=O)c3ccc(C#N)cc3)ccc21] | 4-cyano-N-(1-ethyl-2-oxo-6- quinolyl)benzenesulfonamide |
| 38 | 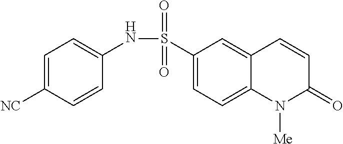 [SMILES: Cn1c(=O)ccc2cc(S(=O)(=O)Nc3ccc(C#N)cc3)ccc21] | N-(4-cyanophenyl)-1-methyl-2-oxo- quinoline-6-sulfonamide |
| 45 | 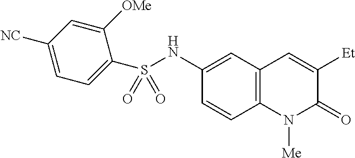 [SMILES: CCc1cc2cc(NS(=O)(=O)c3ccc(C#N)cc3OC)ccc2n(C)c1=O] | 4-cyano-N-(3-ethyl-1-methyl-2-oxo-6- quinolyl)-2-methoxy- benzenesulfonamide |
| 49 | 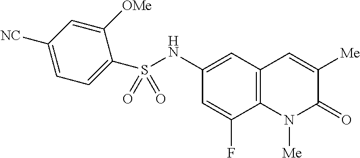 [SMILES: COc1cc(C#N)ccc1S(=O)(=O)Nc1cc(F)c2c(c1)cc(C)c(=O)n2C] | 4-cyano-N-(8-fluoro-1,3-dimethyl-2-oxo- 6-quinolyl)-2-methoxy- benzenesulfonamide |
| 50 | 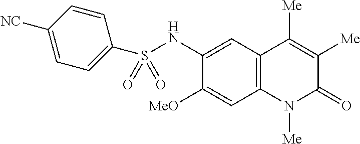 [SMILES: COc1cc2c(cc1NS(=O)(=O)c1ccc(C#N)cc1)c(C)c(C)c(=O)n2C] | 4-cyano-N-(7-methoxy-1,3,4-trimethyl- 2-oxo-6-quinolyl)benzenesulfonamide |
| 51 | 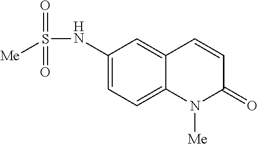 [SMILES: Cn1c(=O)ccc2cc(NS(C)(=O)=O)ccc21] | N-(1-methyl-2-oxo-6- quinolyl)methanesulfonamide |
| 52 | 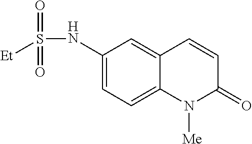 [SMILES: CCS(=O)(=O)Nc1ccc2c(ccc(=O)n2C)c1] | N-(1-methyl-2-oxo-6- quinolyl)ethanesulfonamide |
| 53 | 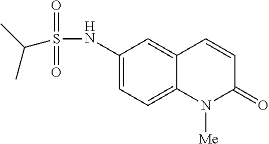 [SMILES: CC(C)S(=O)(=O)Nc1ccc2c(ccc(=O)n2C)c1] | N-(1-methyl-2-oxo-6-quinolyl)propane- 2-sulfonamide |
| 54 | 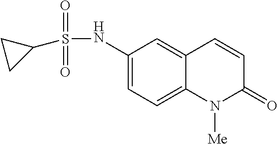 [SMILES: Cn1c(=O)ccc2cc(NS(=O)(=O)C3CC3)ccc21] | N-(1-methy1-2-oxo-6- quinolyl)cyclopropanesulfonamide |
| 55 | 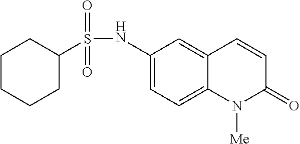 [SMILES: Cn1c(=O)ccc2cc(NS(=O)(=O)C3CCCCC3)ccc21] | N-(1-methyl-2-oxo-6-quinolyl) cyclohexanesulfonamide |
| 56 | 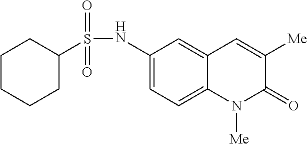 [SMILES: Cc1cc2cc(NS(=O)(=O)C3CCCCC3)ccc2n(C)c1=O] | N-(1,3-dimethyl-2-oxo-6- quinolyl)cyclohexanesulfonamide |
| 59 | 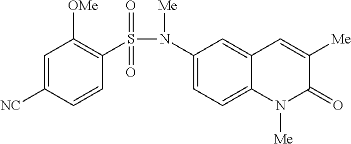 [SMILES: COc1cc(C#N)ccc1S(=O)(=O)N(C)c1ccc2c(c1)cc(C)c(=O)n2C] | 4-cyano-N-(1,3-dimethyl-2-oxo-6- quinolyl)-2-methoxy-N-methyl- benzenesulfonamide. |

23-26. (canceled)

27. A method of treating cancer comprising administering an effective amount of a compound according to claim 1 to a subject.

28. A method according to claim 27 wherein the cancer is characterised by activation of the BRPF1/HOX pathway.

29. A method according to claim 27 wherein the cancer is acute myeloid leukemia (AML).

30. A pharmaceutical composition comprising a compound according to claim 1 and a pharmaceutically acceptable carrier, diluent, or excipient.
